(19) United States
(12) Patent Application Publication
(10) Pub. No.: US 2024/0335810 A1
(43) Pub. Date: Oct. 10, 2024

(54) AEROGELS, METHODS FOR THEIR PREPARATION AND USES THEREOF

(71) Applicant: THE UNIVERSITY OF BRITISH COLUMBIA, Vancouver (CA)

(72) Inventors: Feng Jiang, Vancouver (CA); Hengfei Qin, Vancouver (CA); Ivan Zhang, Vancouver (CA)

(21) Appl. No.: 18/294,036

(22) PCT Filed: Aug. 4, 2022
(86) PCT No.: PCT/CA2022/051190

§ 371 Date: Jan. 31, 2024

Related U.S. Application Data
(60) Provisional application No. 63/230,312, filed on Aug. 6, 2021.

(51) Int. Cl. B01J 13/00; B01J 20/24; B01J 20/28; C08J 9/28

(52) U.S. Cl. CPC B01J 13/0091; B01J 20/24; B01J 20/28011; B01J 20/28045; B01J 20/28047; B01J 20/28054; C08J 9/28; C08J 2205/026; C08J 2301/02

(57) ABSTRACT

The present disclosure relates to aerogels (e.g., superelastic and/or superhydrophobic aerogels), methods for preparation of such aerogels and uses of such aerogels. For example, the methods for preparation can comprise: freezing a suspension comprising nanofibrils and a solvent at a first temperature; freeze-drying the frozen suspension to obtain sub-micron fibers; freezing an aqueous suspension comprising the sub-micron fibers at a second temperature; and freeze-drying the frozen suspension to obtain the aerogel.

### CROSS-REFERENCE TO RELATED APPLICATIONS

The present disclosure claims the benefit of priority from co-pending U.S. provisional application No. 63/230,312 filed on Aug. 6, 2021, the contents of which are incorporated herein by reference in their entirety.

### FIELD

The present disclosure relates to aerogels (e.g., superelastic and/or superhydrophobic aerogels), methods for preparation of such aerogels and uses of such aerogels.

### BACKGROUND

As the lightest solid material, aerogel has demonstrated increasing application potentials ranging from thermal regulation (Wang et al., 2019; Song et al., 2020), energy harvesting and storage (Chen et al., 2020; Qian et al., 2019), sensors (L. Wang et al., 2020; Liu et al., 2021), environmental remediation (Jiang et al., 2018; Jiang & Hsieh, 2014a), and biomedical applications (Najberg et al., 2020; Chen et al., 2016). Such diverse applications of aerogel are ascribed to its numerous advantages including but not limited to low density, high porosity, high specific surface area, low thermal conductivity, and/or biocompatibility. Among various physical properties of aerogel, mechanical response is of great interest as it can limit the application of aerogels. Tremendous amounts of effort have been exerted in improving the mechanical properties of aerogel, primarily focusing on enhancing the compressibility and elasticity performance (Wang et al., 2019; Song et al., 2020; Chen et al., 2020; Qian et al., 2019; L. Wang et al., 2020; Liu et al., 2021; Jiang et al., 2018; Jiang & Hsieh, 2014a; Najberg et al., 2020; Chen et al., 2016; Meador et al., 2005; Huang et al., 2019; L. Y. Wang et al., 2019). Elastic aerogels, due, for example, to the high resilience, elasticity, and shape-recovery performance, have been actively researched, as the ability to recover from large compressive strain is desirable for various applications such as electrical signal sensing (Liu et al., 2021), water treatment (D. Wang et al., 2020), thermal and acoustic insulation (Jia et al., 2020), air filtration (Li et al, 2020), and/or energy storage (Chen et al., 2020).

Superelastic aerogels can be realized by taking advantage of the inherent superelasticity of graphitic carbon (Qiu et al., 2012; Wu et al., 2015), reconstructed polymeric (Cho et al., 2018; Wu et al., 2020) and ceramic sub-micrometer (sub-micron) wide fibers (Wang et al., 2017; Si et al., 2018; Si et al., 2014). Other than the intrinsic elasticity, continuous sub-micron fibers can also introduce structural continuity and crosslinked networks to provide additional elasticity to aerogels (Zhang et al., 2020; Dou et al., 2020; F. Wang et al., 2020). Superelastic polymeric and inorganic aerogels have therefore been realized by firstly constructing elastic sub-micron fibers using solution blow spinning (Jia et al., 2020) or electrospinning (Si et al., 2014), followed by assembling into aerogel via an ice-templating method to create hierarchical lamellar elastic structures (Si et al., 2018). However, complex synthesis processes and high process complexity involved for fiber spinning could potentially limit the applications of these superelastic aerogels, especially from materials that cannot be easily spun into fibers.

Bio-derived natural polymers, especially cellulose, have attracted great interest as building blocks for aerogel, owing to their ready availability and ease of processing (Li et al., 2021: Ferreira et al., 2021). Conventional cellulose aerogel synthesis involves a dissolution-regeneration process using large amounts of solvent (Jin et al., 2004; Cai et al., 2008). Lately, nanocellulose, especially cellulose nanofibrils (CNFs), has been widely used to construct aerogel with a high specific surface and excellent mechanical properties (Zeng et al., 2021; Sakuma et al., 2021; Chen et al., 2021). Due to the high surface area and abundant polar functional groups, ultra-lightweight aerogel can be facilely prepared by freeze-drying aqueous CNF suspensions, showing excellent structural integrity (Jiang et al., 2018; Jiang & Hsieh, 2014a). Although CNF aerogel shows good compressibility that can be compressed over 90% strain without collapsing, the compressed aerogel generally shows poor elasticity and cannot recover from the compressed state. The lack of elasticity can be ascribed to both non-elastic microstructure and strong hydrogen bonding formed between adjacent CNFs during compression. Elastic CNF aerogel has been prepared by a unidirectional freezing method, but the prepared aerogel showed anisotropic elasticity with unsatisfactory elastic performance along the longitudinal direction (Chen et al., 2019; Zhang et al., 2019). Elastic cellulose aerogel has also been prepared via a top-down process by selective removal of lignin and hemicellulose from wood, but the aerogel only showed anisotropic elastic performance along the radial direction (Song et al., 2018; Guan et al., 2018). However, no effective strategy has been reported in designing an isotropic superelastic CNF aerogel.

### SUMMARY

The methods disclosed herein can be used to prepare, for example, superelastic aerogels with fast shape recovery performance from large compressive strain. This is highly desired for numerous applications such as but not limited to thermal insulation in clothing, high-sensitive sensors, and oil contaminant removal. Fabrication of superelastic cellulose nanofibrils aerogel is challenging as the CNF can assemble into non-elastic sheet-like cell walls. A dual ice-templating assembly (DITA) strategy was used that can control the assembly of CNF into sub-micron fibers by extremely low temperature freezing (e.g., approximately −196° C.), which can further assemble into elastic aerogel with interconnected sub-micron fibers by freezer freezing (e.g., approximately −20° C.) and freeze drying. A CNF aerogel prepared from the DITA process demonstrated isotropic superelastic behavior that can recover from over 80% compressive strain along both longitudinal and cross-sectional directions, even under an extremely cold liquid nitrogen environment. The elastic CNF aerogel was facilely modified by chemical vapor deposition of organosilane, demonstrating superhydrophobicity (164° water contact angle), high liquid absorption (489 g/g of chloroform absorption capacity), self-cleaning, thermal insulating (0.023 W/(m·K)), and infrared (IR) shielding properties. This new DITA strategy provides a facile design of superelastic aerogel from bio-based nanomaterials, and the derived high performance multifunctional elastic aerogel is expected to be useful for wide-range applications.

Accordingly, the present disclosure includes a method of preparing an aerogel, the method comprising:
freezing a suspension comprising nanofibrils and a solvent at a first temperature;
freeze-drying the frozen suspension to obtain sub-micron fibers;
freezing an aqueous suspension comprising the sub-micron fibers at a second temperature; and
freeze-drying the frozen suspension to obtain the aerogel.

In an embodiment, the solvent in the suspension comprising the nanofibrils is water, tert-butanol, dimethyl sulfoxide or mixtures thereof. In another embodiment, the suspension comprising nanofibrils is an aqueous suspension comprising the nanofibrils in an amount of from about 0.01 wt % to about 1 wt %, based on the total weight of the aqueous suspension. In a further embodiment, the aqueous suspension comprises the nanofibrils in an amount of about 0.05 wt %, based on the total weight of the aqueous suspension.

In an embodiment, the aqueous suspension comprising the sub-micron fibers comprises the sub-micron fibers in an amount of from about 0.01 wt % to about 2 wt %, based on the total weight of the aqueous suspension. In another embodiment, the aqueous suspension comprises the sub-micron fibers in an amount of from about 0.2 wt % to about 2 wt %, based on the total weight of the aqueous suspension. In a further embodiment, the aqueous suspension comprises the sub-micron fibers in an amount of about 0.2 wt %.

In an embodiment, the nanofibrils comprise cellulose, chitin, protein or combinations thereof. In another embodiment, the nanofibrils comprise cellulose nanofibrils.

In an embodiment, the cellulose nanofibrils are prepared by a method comprising mechanical dispersion; enzymatic hydrolysis and mechanical dispersion; or chemical modification and mechanical dispersion. In another embodiment, the cellulose nanofibrils are oxidized cellulose nanofibrils prepared by a method comprising oxidizing a cellulose source and mechanically dispersing the oxidized cellulose source. In a further embodiment, the oxidation of the cellulose source comprises 2,2,6,6-tetramethylpiperidine 1-oxyl (TEMPO) oxidation.

In an embodiment, the second temperature is greater than the first temperature.

In an embodiment, the first temperature is in the range of from about −80° C. to about −200° C. In another embodiment, the first temperature is about −196° C.

In an embodiment, the second temperature is in a range of about −4° C. to about −50° C. In another embodiment, the second temperature is about −20° C.

In an embodiment, the method further comprises modifying a surface of the aerogel with a hydrophobic surface modifying agent. In another embodiment, the modifying the surface comprises chemical vapor deposition of methyltrimethoxysilane. In a further embodiment, the modified surface is superhydrophobic.

The present disclosure also includes an aerogel prepared by a method of preparing an aerogel as described herein.

The present disclosure also includes an aerogel comprising a honeycomb structure, the cell walls of the honeycomb comprising interconnected sub-micron fibers.

In an embodiment, the sub-micron fibers comprise cellulose, chitin, protein or combinations thereof. In another embodiment, the sub-micron fibers are cellulose sub-micron fibers. In a further embodiment, the sub-micron fibers comprise oxidized cellulose. In an embodiment, at least a portion of the cellulose has a cellulose Iβ crystal structure.

In an embodiment, a surface of the aerogel is modified with a hydrophobic surface modifying agent. In another embodiment, the hydrophobic surface modifying agent comprises methyltrimethoxysilane. In a further embodiment, the modified surface is superhydrophobic.

In an embodiment, density of the aerogel is from about 2 mg/m³ to about 22 mg/m³. In another embodiment, porosity of the aerogel is from about 98.7% to about 99.8%. In an embodiment, the aerogel has isotropic superelasticity.

The present disclosure also includes a use of an aerogel of the present disclosure (including an aerogel prepared by a method of the present disclosure) for oil-water separation, as an absorbent, as a thermal insulator, as an acoustic insulator, as a drug carrier, as a tissue scaffold, as an infrared shield and/or in a sensor.

The present disclosure also includes a method of preparing sub-micron fibers comprising a cellulose Iβ crystal structure, the method comprising:
freezing a suspension comprising cellulose nanofibrils and a solvent at a temperature of from about −80° C. to about −200° C.; and
freeze-drying the frozen suspension to obtain the sub-micron fibers.

The present disclosure also includes a sub-micron fiber comprising a cellulose Iβ crystal structure prepared by a method of preparing sub-micron fibers comprising a cellulose Iβ crystal structure as described herein.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

FIG. 1 is a conductometric titration curve of the CNF according to an example of the present disclosure to determine the surface carboxylate content.

FIG. 2 is a schematic illustration of the assembly process for a conventional CNF aerogel. A one-step ice-templating assembly process at −20° C. is used to fabricate the conventional CNF aerogel, showing a honeycomb porous structure with film-like cell walls (bottom images). Scale bars show 500 nm (upper image); 500 μm (bottom left image); and 20 μm (bottom right image).

FIG. 3 shows no shape recovery after compressing for a conventional CNF aerogel fabricated by a one-step ice-templating assembly process at −20° C.

FIG. 4 is a schematic illustration of the assembly process for a superelastic cellulose nanofibrils aerogel. A dual ice-templating assembly (DITA) process at −196° C. and −20° C. to fabricate the superelastic CNF aerogel, showing a honeycomb porous structure with interconnected sub-micron fibers cell walls (bottom images) Scale bars show 500 nm (upper, middle and bottom right images) and 500 μm (bottom left image).

FIG. 5 shows an exemplary ultraviolet-visible (UV-Vis) transmittance spectrum of a 0.05% CNF suspension.

FIG. 6 is a plot showing the size distribution of sub-micron CNF fibers prepared by freezing a dilute CNF suspension (0.05 wt %) at −196° C. followed by freeze-drying.

FIG. 7 shows field-emission scanning electron microscope (FE-SEM) images of a 0.2 wt % superelastic CNF aerogel according to an example of the present disclosure at different magnification scales. Scale bars show 100 μm (upper left image); 20 μm (upper right image); 5 μm (lower left image); and 1 μm (lower right image).

FIG. 8 shows instantaneous shape recovery after compressing along the longitudinal direction for an aerogel fabricated by a DITA process.

FIG. 9 shows an exemplary X-ray powder diffraction (XRD) spectrum of assembled sub-micron CNF fibers.

FIG. 10 is a plot showing density (left column for each concentration) and porosity (right column for each concentration) of the sub-micron CNF aerogels.

FIG. 11 shows N2 adsorption-desorption isotherms of sub-micron CNF fibers and superelastic CNF aerogels.

FIG. 12 shows pore size distribution of sub-micron CNF fibers and superelastic CNF aerogels.

FIG. 13 shows photographs of a large piece of superelastic CNF aerogel (length: 180 mm, width: 120 mm) demonstrating great flexibility that can be rolled and folded.

FIGS. 14-16 show hydrophobization of CNF aerogel via chemical vapor deposition: FIG. 14 shows a schematic illustration of vapor deposition of methyltrimethoxysilane (MTMS) on CNF aerogel wherein water is vaporized in a reactor to promote the hydrolysis of MTMS: FIG. 15 shows Fourier-transform infrared spectroscopy (FTIR) spectra of CNF aerogel before (*) and after (**) silanization (MTMS-CNF aerogel) (top); and an energy-dispersive X-ray spectroscopy (EDS) spectrum with weight concentration for C, O, and Si (bottom); and FIG. 16 shows EDS elemental C (top; red in color image), O (middle: green in color image), and Si (bottom; blue in color image) mapping images of MTMS-CNF aerogel. Brightness increased by 40% and contrast by 20% to improve clarity in greyscale images.

FIGS. 17-20 show hydrophobic and oil absorption properties of MTMS-CNF aerogel: FIG. 17 shows a digital image of various colored aqueous solutions (methylene blue, vinegar, coffee, soy sauce, potassium dichromate, and methyl orange) and chloroform (dyed with methyl red to enhance visual contrast) deposited on the surface of MTMS-CNF aerogel (top); digital images of water contact angle on the surface of MTMS-CNF aerogel over different time periods (bottom left), and the evolution of water contact angle over time (bottom right); FIG. 18 shows photographs of water contact angles of superelastic CNF aerogel: top (left images), side (middle images) and interior (right images); FIG. 19 shows selective absorption of chloroform (dyed with methyl red) from the bottom of water (top); and screen shots of self-cleaning performance of the MTMS-CNF aerogel, showing dust can be cleared off the aerogel surface by water at inclination angle of 20° (bottom); and FIG. 20 is a plot showing the absorption capacity (weight gain, %) of MTMS-CNF aerogel towards different types of oil and organic solvents, from top to bottom: hexadecane, toluene, hexane, tetrahydrofuran, acetone, chloroform, canola oil, silicone oil and pump oil.

FIGS. 21-27 show mechanical properties of superelastic CNF aerogel under compression: FIG. 21 shows stress-strain curves of CNF aerogels with different initial concentrations of 0.2, 0.4, 0.6, 0.8, 1 and 2 wt % (top); and stress-strain curves of CNF aerogels with different initial concentrations of 0.2, 0.4, and 0.6 wt % (bottom); FIG. 22 shows Young's modulus (left column for each concentration) and yield stress (right column for each concentration); FIG. 23 is a digital image showing the rigid/strong behavior of the 2% CNF aerogel (0.186 g) under 1 kg weight; FIG. 24 shows cyclic stress-strain curves of 0.2 wt % (top) and 0.6 wt % (bottom) superelastic CNF aerogel under different strains; FIG. 25 shows cyclic compressive stress-strain curves at 40% maximum strain for 0.2% superelastic CNF aerogel with 50 mm/min loading and unloading rates (top); and the ultimate stress at 40% strain and energy loss coefficient curves (1-50 cycles) of 0.2% superelastic CNF aerogel (bottom); FIG. 26 shows cyclic compressive stress-strain curves at 40% maximum strain for 0.6% superelastic CNF aerogel (top); and the ultimate stress at 40% strain and energy loss coefficient curves (1-50 cycles) of 0.6% superelastic CNF aerogel (bottom); and FIG. 27 shows digital images showing the compressibility of the superelastic aerogel under 200 g weights (upper images); and digital images showing excellent elasticity of the 0.2% CNF aerogel immersed in liquid N2, showing instantaneous shape recover from over 80% strain (lower images).

FIGS. 28-30 show thermal properties of superelastic CNF aerogel: FIG. 28 shows the thermal conductivity of aerogel at different initial CNF concentrations (upper plot); and the temperature-versus-time curves of 0.2% CNF aerogel (height: 8 mm) on a hot plate (lower left plot) and ice (lower right plot), wherein the black dashed line in the lower plots represents room temperature; large circular dots in the lower left plot represent the surface temperature of aerogel on the hot plate; large circular dots in the lower right plot represent the surface temperature of aerogel on ice; and the triangles represent the temperature of the hot plate (lower left plot) or ice (lower right plot); FIG. 29 shows infrared images of CNF aerogel (thickness: 8 mm) on top of 80° C. hot plate (upper image), −17° C. ice block (middle image), and human palm (bottom image); and FIG. 30 shows infrared images of 0.2% CNF aerogel (height: 19 mm) on the hot plate during the course of 150 min. The last infrared image is the temperature at the top surface of the aerogel at 150 min.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps. As used herein, the word “consisting” and its derivatives, are intended to be close ended terms that specify the presence of stated features, elements, components, groups, integers, and/or steps, and also exclude the presence of other unstated features, elements, components, groups, integers and/or steps. The term “consisting essentially of” and any form thereof, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₆alkyl means an alkyl group having 1, 2, 3, 4, 5 or 6 carbon atoms.

The term “suitable” as used herein means that the selection of the particular compound, material and/or conditions would depend on the specific synthetic manipulation to be performed, and/or the identity of the compound(s) to be transformed, but the selection would be well within the skill of a person skilled in the art. All method steps described herein are to be conducted under conditions sufficient to provide the product shown.

The term “superhydrophobic” as used herein refers to a surface with a water droplet static contact angle above 150°. The term “static contact angle” as used herein refers to the contact angle of a static drop on a surface.

The term “superelastic” as used herein refers to an aerogel capable of undergoing large deformation (e.g., a compression strain of at least 50% to 80% strain or greater) and at least substantially, optionally completely recover the deformation during the unloading phase substantially immediately, for example in a time of less than 10, 5, or 3 seconds.

The term “nanofibril” as used herein refers to a fiber of a material, typically having an elongated form (but may also, in some embodiments, include suitable other forms such as suitable particles), wherein the average diameter is 100 nm or less. In an embodiment, the average diameter of the nanofibrils is about 1 nm to about 50 nm. In another embodiment the average diameter of the nanofibrils is about 2 nm to about 10 nm. In a further embodiment, the average diameter of the nanofibrils is about 3 nm to about 5 nm. In contrast to known methods for preparing elastic aerogels, the methods of the present disclosure can be used to prepare elastic aerogels from nanofibrils with lower aspect ratios. The term “nanofibril” as used herein includes materials that are completely in the form of nanofibrils but may also include materials with minor amounts of material in non-nanofibril form: e.g., materials that consist essentially of nanofibrils but also include material in non-nanofibril form.

The term “sub-micron fiber” as used herein refers to a fiber of a material having an elongated form, wherein at least one diameter is greater than 100 nm and less than 1000 nm. In an embodiment, the average diameter is from about 100 nm to about 200 nm, about 100 nm to about 170 nm, or about 100 nm to 500 nm. The term “sub-micron fiber” as used herein includes materials that are completely in the form of sub-micron fibers but may also include materials with minor amounts of material outside of that form: e.g., materials that consist essentially of sub-micron fibers but also include material outside of that form such as fibers having average diameters of less than 100 nm. In an embodiment, the sub-micron fibers comprise less than about 25 wt %, about 20 wt %, or about 17.5 wt % of material having average diameters of less than 100 nm. In an embodiment, the sub-micron fibers comprise material having average diameters in the range of about 80 nm to less than 100 nm.

### II. Methods

A dual ice-templating assembly (DITA) process was used for the fabrication of a lightweight and superelastic CNF aerogel with ultralow density (as low as 2 mg/cm³), excellent isotropic elasticity and shape recovery at both ambient conditions and in extremely cold environments (−196° C.), as well as low thermal conductivity (0.023 W/(m·K)). In addition, superhydrophobic CNF aerogel with a contact angle of 164° was facilely prepared by chemical vapor deposition with organosilane. The superhydrophobic CNF aerogel showed superabsorbancy towards oil and organic solvent (up to 489 times of its own weight) and efficient oil/water separation and self-cleaning performance. The proposed DITA strategy is proven to be a facile approach in developing superelastic CNF aerogel without chemical crosslinking, and is expected to be extended to fabricate aerogel from other bio-based nanomaterials.

Accordingly, the present disclosure includes a method of preparing an aerogel, the method comprising:
freezing a suspension comprising nanofibrils and a solvent at a first temperature; freeze-drying the frozen suspension to obtain sub-micron fibers;
freezing an aqueous suspension comprising the sub-micron fibers at a second temperature; and
freeze-drying the frozen suspension to obtain the aerogel.

The suspension comprising the nanofibrils can comprise any suitable solvent or combination thereof. In an embodiment, the solvent comprises, consists essentially of or consists of water, tert-butanol, dimethyl sulfoxide or combinations thereof. In another embodiment, the suspension comprising nanofibrils is an aqueous suspension comprising the nanofibrils. The concentration of the nanofibrils in the suspension (e.g., the aqueous suspension) is any suitable concentration. In an embodiment, the suspension comprises the nanofibrils in an amount of from about 0.01 wt % to about 1 wt %, based on the total weight of the suspension. In another embodiment, the suspension comprises the nanofibrils in an amount of about 0.01 wt % to about 0.1 wt %, based on the total weight of the suspension. In a further embodiment, the suspension comprises the nanofibrils in an amount of about 0.05 wt %, based on the total weight of the suspension. In an embodiment, the suspension comprising nanofibrils is an aqueous suspension comprising the nanofibrils in an amount of from about 0).01 wt % to about 1 wt %, based on the total weight of the aqueous suspension. In another embodiment, the aqueous suspension comprises the nanofibrils in an amount of about 0.01 wt % to about 0).1 wt %, based on the total weight of the aqueous suspension. In a further embodiment, the aqueous suspension comprises the nanofibrils in an amount of about 0.05 wt %, based on the total weight of the aqueous suspension.

The concentration of the sub-micron fibers in the aqueous suspension is any suitable concentration. For example, in some embodiments, the aqueous suspension comprises the sub-micron fibers in an amount suitable to achieve a homogeneous suspension. For example, in an embodiment, the aqueous suspension comprising the sub-micron fibers comprises the sub-micron fibers in an amount of less than about 2 wt %, based on the total weight of the aqueous suspension. In another embodiment, the aqueous suspension comprising the sub-micron fibers comprises the sub-micron fibers in an amount of from about 0.01 wt % to about 2 wt %, based on the total weight of the aqueous suspension. In a further embodiment, the aqueous suspension comprises the sub-micron fibers in an amount of from about 0.2 wt % to about 2 wt %, based on the total weight of the aqueous suspension. It will be appreciated by a person skilled in the art having regard to the present disclosure that the properties of the aerogels such as the mechanical performance of the aerogels produced by the methods of the present disclosure may vary, for example, based on the concentration of the sub-micron fibers in the aqueous suspension. For example, an aerogel prepared using a concentration of 2 wt % of cellulose sub-micron fibers demonstrated rigid behaviour whereas an aerogel prepared using a concentration of 0.2 wt % cellulose sub-micron fibers demonstrated superelastic behaviour. In an embodiment, the aqueous suspension comprising the sub-micron fibers comprises the sub-micron fibers in an amount of about 0.1 wt % to about 0.3 wt % or about 0.2 wt %. In another embodiment, the aqueous suspension comprises the sub-micron fibers in an amount of about 1 wt % to about 3 wt % or about 2 wt %. In a further embodiment, the aqueous suspension comprises the sub-micron fibers in an amount of 0.2%, 0.4%, 0.6%, 0.8%, 1.0%, or 2.0%.

The term “aqueous” as used herein in reference to a suspension includes suspensions wherein the solvent consists of water but also includes aqueous suspensions that include minor amounts of another solvent or mixtures thereof, for example, less than about 5 wt %, 4 wt %, 3 w1%, 2 wt %, 1 wt %, 0.5 wt %, 0.1 wt %, 0.05 wt % or 0.01 wt % of the other solvent or mixture thereof so long as the presence of the other solvent or mixture thereof still allows for suitable conditions for obtaining the sub-micron fibers or aerogel, as the case may be.

The nanofibrils can comprise any suitable material or combination thereof. In an embodiment, the nanofibrils comprise a bio-based material such as cellulose, chitin, protein or combinations thereof. In another embodiment, the nanofibrils comprise cellulose nanofibrils. In a further embodiment, the nanofibrils comprise chitin nanofibrils. In another embodiment, the nanofibrils comprise protein nanofibrils. In a further embodiment, the nanofibrils comprise combinations of cellulose nanofibrils, chitin nanofibrils and protein nanofibrils.

Suitable nanofibrils can be prepared using known methods and/or means, the selection of which can readily be made by a person skilled in the art. For example, suitable methods for preparing cellulose nanofibrils can comprise chemical and/or biological treatment (e.g., enzymatic hydrolysis) and/or mechanical dispersion of a suitable cellulose source. In an embodiment, the cellulose nanofibrils are prepared by a method comprising mechanical dispersion: enzymatic hydrolysis and mechanical dispersion; or chemical modification and mechanical dispersion. The mechanical dispersion can comprise any suitable method and/or means, the selection of which can also be readily made by a person skilled in the art. For example, in an embodiment, the mechanical dispersion comprises blending (e.g., high-speed blending), homogenizing (e.g., high-pressure homogenizing), microfluidizing, disc grinding, sonication or combinations thereof. In another embodiment, the mechanical dispersion comprises high-speed blending.

In some embodiments, the cellulose nanofibrils are oxidized cellulose nanofibrils. The term “oxidized” as used herein in reference to cellulose nanofibrils refers to cellulose nanofibrils wherein at least a portion of the hydroxyl groups in cellulose chains have been converted to carboxyl groups or a group comprising a carboxyl group (e.g., a carboxymethyl group). Methods for preparing oxidized cellulose nanofibrils are well known in the art and a suitable method can be readily selected by a person skilled in the art.

In an embodiment, the oxidized cellulose nanofibrils are prepared by a method comprising oxidizing a cellulose source and mechanically dispersing the oxidized cellulose source. In an embodiment, the oxidation of the cellulose source comprises 2,2,6,6-tetramethylpiperidine 1-oxyl (TEMPO) oxidation. Methods for oxidation of cellulose with nitroxyl radicals are well known in the art (see, for example: Isogai et al., 2018) and the selection of suitable conditions for preparation of a particular oxidized cellulose nanofibril can be readily made by a person skilled in the art. In some embodiments, the TEMPO oxidation comprises addition of a suitable amount of NaClO as a primary oxidant. In some embodiments, prior to freezing the suspension comprising the nanofibrils, the method further comprises diluting the suspension obtained subsequent to oxidation and mechanical dispersion to a suitable concentration (e.g., about 0.1 wt % to about 0.3 wt % or about 0.2 wt %, based on the total weight of the suspension), centrifuging the suspension at a suitable speed (e.g., about 1500 rpm to about 2500 rpm or about 2000 rpm) for a suitable time (e.g., about l minute to about 20 minutes or about 10 minutes) and discarding the precipitate.

The cellulose source can be any suitable cellulose source, the selection of which can be made by a person skilled in the art. For example, in an embodiment, cellulose raw material is treated to substantially remove non-cellulosic components to obtain the cellulose source. Methods for treating cellulose raw material to remove non-cellulosic components are well known in the art and may depend, for example, on the particular cellulose raw material. For example, plant-derived cellulose raw materials typically comprise cellulose in admixture with other substances such as hemicellulose, lignin and pectin. In contrast, other sources of cellulose raw materials such as bacteria and/or algae comprise cellulose with an already higher degree of purity. A suitable method for treating cellulose raw material to remove non-cellulosic components can be readily selected by a person skilled in the art. Alternatively, suitable cellulose sources may be commercially available and selected by the skilled person.

In an embodiment, the cellulose raw material is wood-based (e.g., from a softwood tree such as pine, fir, larch, spruce, cypress or combinations thereof or a hardwood tree such as birch, poplar, basswood, eucalyptus, maple or combinations thereof) or from other plant sources such as straw; bagasse, bamboo, reed, jute, wheat, cotton, rice, flax or combinations thereof, or a non-plant based source such as bacteria and/or algae or combinations thereof. In another embodiment, the cellulose source comprises a suitable wood pulp such as but not limited to Northern bleached softwood kraft (NBSK), Southern bleached softwood kraft (SBSK) or combinations thereof. In a further embodiment, the cellulose source comprises, consists essentially of or consists of northern bleached softwood kraft (NBSK).

In some embodiments, the second temperature may be less than or equal to the first temperature, provided that conditions remain suitable for obtaining the sub-micron fibers and aerogel. However, freezing at a lower temperature is more energy intensive. Accordingly, even if a lower temperature is suitable to use for the second temperature, it may be desirable to have a higher temperature. In an embodiment, the second temperature is greater than the first temperature. The first temperature can be any suitable temperature for allowing for the assembly of the nanofibrils into sub-micron fibers. For example, a person skilled in the art having regard to the present disclosure would readily appreciate that the conditions for obtaining the sub-micron fibers including, for example, the first temperature should be selected such that growth of large ice crystals is inhibited. In an embodiment, the first temperature is in the range of from about −80° C. to about −200° C. In another embodiment, the first temperature is in the range of from about −180° C. to about −210° C. or about −196° C. In an embodiment, the suspension is frozen at the first temperature for a time of about 5 minutes to about 2 hours or about 30 minutes. The conditions for freeze-drying the frozen suspension comprising the nanofibrils are any suitable conditions, the selection of which can be readily made by a person skilled in the art. In an embodiment, the frozen suspension is freeze-dried to obtain the sub-micron fibers at a temperature of about −20° C. to about −75° C. or about −50° C. at a pressure of about 0.05 mBar to about 0.2 mBar or about 0.12 mBar for a time of about 6 hours to about 4 days or about 48 hours, optionally a time until substantially all of the solvent is removed. The temperature of the freeze-dried sub-micron fibers is raised and/or allowed to rise to a third temperature that is greater than the temperature used for the freeze drying prior to being suspended in the aqueous suspension comprising the sub-micron fibers. In an embodiment, the third temperature is ambient temperature (e.g., a temperature of from about 4° C. to about 40° C. or about 25° C.). In another embodiment, the freeze-dried sub-micron fibers are stored at the third temperature prior to being suspended in the aqueous suspension comprising the sub-micron fibers.

The conditions for obtaining the aerogel including, for example, the second temperature can be any suitable conditions allowing for the sub-micron fibers to be interconnected and an aerogel having pores (e.g., comprising a honeycomb structure) to be obtained. For example, a person skilled in the art having regard to the present disclosure would readily appreciate that the conditions for obtaining the aerogel including, for example, the second temperature should be selected such that ice crystals of a suitable size for templating such pores are formed. In an embodiment, the second temperature is in the range of from about −4° C. to about −50° C. In another embodiment, the second temperature is in the range of from about −10° C. to about −30° C. or about −20° C. In an embodiment, the suspension is frozen at the second temperature for a time of about 30 minutes to about 24 hours or about 3 hours. The conditions for freeze-drying the frozen suspension comprising the sub-micron fibers are any suitable conditions, the selection of which can be readily made by a person skilled in the art. In an embodiment, the frozen suspension is freeze-dried to obtain the aerogel at a temperature of about −20° C. to about −75° C. or about −50° C. at a pressure of about 0.05 mBar to about 0.2 mBar or about 0.12 mBar for a time of about 6 hours to about 4 days or about 48 hours, optionally a time until substantially all of the solvent is removed.

In an embodiment, the method further comprises modifying a surface of the aerogel with a hydrophobic surface modifying agent. The term “hydrophobic surface modifying agent” as used herein refers to an agent that can be used to modify a surface of the aerogel such that the surface is less hydrophilic (i.e., has greater hydrophobicity) than the surface of a corresponding aerogel that has not been modified by the agent. The hydrophobic surface modifying agent can be any suitable hydrophobic surface modifying agent and can be readily selected by a person skilled in the art. In an embodiment, the hydrophobic surface modifying agent is a suitable silane, such as a silane of the Formula (I):

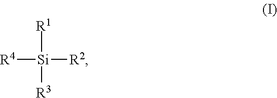

wherein R¹ is C₁₋₆alkyl and R², R³ and R⁴ are each independently chosen from C₁₋₆alkyl, hydroxy and a group that is hydrolysable under conditions to modify the surface, provided that at least one of R², R³ and R⁴ is a hydroxy or a group that is hydrolysable under conditions to modify the surface. A person skilled in the art would readily understand what groups are hydrolysable under particular conditions used to modify the surface and would be able to select a suitable silane and conditions accordingly. In an embodiment, R¹ is C₁₋₆ alkyl and R², R³ and R⁴ are each independently C₁₋₆alkoxy. In another embodiment, R¹ is methyl. In a further embodiment, R², R³ and R⁴ are each methoxy. In an embodiment, the hydrophobic surface modifying agent is methyltrimethoxysilane. In an embodiment, the modifying the surface comprises chemical vapor deposition of the hydrophobic surface modifying agent (e.g., the suitable silane such as methyltrimethoxysilane). However, any suitable method for modifying the surface with a hydrophobic surface modifying agent may be used such as solution-based methods. In another embodiment, the modified surface is superhydrophobic. In another embodiment, the water contact angle of the modified surface is greater than about 155°.

The temperature of the freeze-dried aerogel is typically raised and/or allowed to rise to a fourth temperature that is greater than the temperature used for the freeze drying prior to modification a surface of the aerogel with the hydrophobic surface modifying agent. In an embodiment, the fourth temperature is ambient temperature (e.g., a temperature from about 4° C. to about 40° C. or about 25° C.). In another embodiment, the freeze-dried aerogel is stored at the fourth temperature prior to modification of the surface of the aerogel.

The CNF sub-micron fibers prepared by freezing a suspension comprising nanofibrils and a solvent in an extremely cold environment and freeze-drying the frozen suspension preserved a cellulose Iβ crystal structure. In contrast, other cellulosic sub-micron fibers prepared, for example, by electrospinning or solution blow spinning, are of a cellulose II crystal allomorph. Accordingly, the present disclosure also includes a method of preparing sub-micron fibers comprising a cellulose Iβ crystal structure, the method comprising:
freezing a suspension comprising cellulose nanofibrils and a solvent at a temperature of from about −80° C. to about −200° C.; and
freeze-drying the frozen suspension to obtain the sub-micron fibers.

In an embodiment, an X-ray powder diffraction (XRD) spectrum of the sub-micron fibers shows peaks at about 14.6°, about 15.4° and about 22.3° 2θ.

The suspension comprising the cellulose nanofibrils can comprise any suitable solvent or combination thereof. In an embodiment, the solvent comprises, consists essentially of or consists of water, tert-butanol, dimethyl sulfoxide or combinations thereof. In another embodiment, the suspension comprising cellulose nanofibrils is an aqueous suspension comprising the nanofibrils. The concentration of the cellulose nanofibrils in the suspension (e.g., the aqueous suspension) is any suitable concentration. In an embodiment, the suspension comprises the cellulose nanofibrils in an amount of from about 0.01 wt % to about 1 wt %, based on the total weight of the suspension. In another embodiment, the suspension comprises the cellulose nanofibrils in an amount of about 0.01 wt % to about 0.1 wt %, based on the total weight of the suspension. In a further embodiment, the suspension comprises the cellulose nanofibrils in an amount of about 0.05 wt %, based on the total weight of the suspension. In an embodiment, the suspension comprising cellulose nanofibrils is an aqueous suspension comprising the cellulose nanofibrils in an amount of from about 0.01 wt % to about 1 wt %, based on the total weight of the aqueous suspension. In another embodiment, the aqueous suspension comprises the cellulose nanofibrils in an amount of about 0.01 wt % to about 0.1 wt %, based on the total weight of the aqueous suspension. In a further embodiment, the aqueous suspension comprises the cellulose nanofibrils in an amount of about 0.05 wt %, based on the total weight of the aqueous suspension.

The cellulose nanofibrils can comprise any suitable cellulose or combination thereof. Suitable cellulose nanofibrils can be prepared using known methods and/or means, the selection of which can readily be made by a person skilled in the art. For example, suitable methods for preparing cellulose nanofibrils can comprise chemical and/or biological treatment (e.g., enzymatic hydrolysis) and/or mechanical dispersion of a suitable cellulose source. In an embodiment, the cellulose nanofibrils are prepared by a method comprising mechanical dispersion: enzymatic hydrolysis and mechanical dispersion: or chemical modification and mechanical dispersion. The mechanical dispersion can comprise any suitable method and/or means, the selection of which can also be readily made by a person skilled in the art. For example, in an embodiment, the mechanical dispersion comprises blending (e.g., high-speed blending), homogenizing (e.g., high-pressure homogenizing), microfluidizing, disc grinding, sonication or combinations thereof. In another embodiment, the mechanical dispersion comprises high-speed blending.

In some embodiments, the cellulose nanofibrils are oxidized cellulose nanofibrils. The term “oxidized” as used herein in reference to cellulose nanofibrils refers to cellulose nanofibrils wherein at least a portion of the hydroxyl groups in cellulose chains have been converted to carboxyl groups or a group comprising a carboxyl group (e.g., a carboxymethyl group). Methods for preparing oxidized cellulose nanofibrils are well known in the art and a suitable method can be readily selected by a person skilled in the art.

In an embodiment, the oxidized cellulose nanofibrils are prepared by a method comprising oxidizing a cellulose source and mechanically dispersing the oxidized cellulose source. In an embodiment, the oxidation of the cellulose source comprises 2,2,6,6-tetramethylpiperidine 1-oxyl (TEMPO) oxidation. Methods for oxidation of cellulose with nitroxyl radicals are well known in the art (see, for example: Isogai et al., 2018) and the selection of suitable conditions for preparation of a particular oxidized cellulose nanofibril can be readily made by a person skilled in the art. In some embodiments, the TEMPO oxidation comprises addition of a suitable amount of NaClO as a primary oxidant. In some embodiments, prior to freezing the suspension comprising the nanofibrils, the method further comprises diluting the suspension obtained subsequent to oxidation and mechanical dispersion to a suitable concentration (e.g., about 0.1 wt % to about 0.3 wt % or about 0.2 wt %. based on the total weight of the suspension), centrifuging the suspension at a suitable speed (e.g., about 1500 rpm to about 2500 rpm or about 2000 rpm) for a suitable time (e.g., about 1 minute to about 20 minutes or about 10 minutes) and discarding the precipitate.

The cellulose source can be any suitable cellulose source, the selection of which can be made by a person skilled in the art. For example, in an embodiment, cellulose raw material is treated to substantially remove non-cellulosic components to obtain the cellulose source. Methods for treating cellulose raw material to remove non-cellulosic components are well known in the art and may depend, for example, on the particular cellulose raw material. For example, plant-derived cellulose raw materials typically comprise cellulose in admixture with other substances such as hemicellulose, lignin and pectin. In contrast, other sources of cellulose raw materials such as bacteria and/or algae comprise cellulose with an already higher degree of purity. A suitable method for treating cellulose raw material to remove non-cellulosic components can be readily selected by a person skilled in the art. Alternatively, suitable cellulose sources may be commercially available and selected by the skilled person.

In an embodiment, the cellulose raw material is wood-based (e.g., from a softwood tree such as pine, fir, larch, spruce, cypress or combinations thereof or a hardwood tree such as birch, poplar, basswood, eucalyptus, maple or combinations thereof) or from other plant sources such as straw, bagasse, bamboo, reed, jute, wheat, cotton, rice, flax or combinations thereof, or a non-plant based source such as bacteria and/or algae or combinations thereof. In another embodiment, the cellulose source comprises a suitable wood pulp such as but not limited to Northern bleached softwood kraft (NBSK), Southern bleached softwood kraft (SBSK) or combinations thereof. In a further embodiment, the cellulose source comprises, consists essentially of or consists of northern bleached softwood kraft (NBSK).

In an embodiment, the temperature is in the range of from about −180° C. to about −210° C. or about −196° C. In another embodiment, the suspension is frozen for a time of about 5 minutes to about 2 hours or about 30 minutes. The conditions for freeze-drying the frozen suspension comprising the cellulose nanofibrils are any suitable conditions, the selection of which can be readily made by a person skilled in the art. In an embodiment, the frozen suspension is freeze-dried to obtain the sub-micron fibers at a temperature of about −20° C. to about −75° C. or about −50° C. at a pressure of about 0.05 mBar to about 0.2 mBar or about 0.12 mBar for a time of about 6 hours to about 4 days or about 48 hours, optionally a time until substantially all of the solvent is removed.

The present disclosure also includes a sub-micron fiber comprising a cellulose Iβ crystal structure prepared by a method of preparing sub-micron fibers comprising a cellulose Iβ crystal structure as described herein.

### III. Aerogels and Uses Thereof

The present disclosure also includes an aerogel prepared by a method of preparing an aerogel as described herein. It will be appreciated by a person skilled in the art that embodiments relating to such aerogels may be varied as discussed above in relation to the methods.

The present disclosure also includes an aerogel comprising a honeycomb structure, the cell walls of the honeycomb comprising interconnected sub-micron fibers.

In an embodiment, the sub-micron fibers are prepared by a method comprising: freezing a suspension comprising nanofibrils and a solvent at a first temperature: and freeze-drying the frozen suspension to obtain sub-micron fibers, as described herein.

The sub-micron fibers can comprise any suitable material or combination thereof. In an embodiment, the sub-micron fibers comprise a bio-based material such as cellulose, chitin, protein or combinations thereof. In another embodiment, the sub-micron fibers are cellulose sub-micron fibers. In a further embodiment, the nanofibrils are chitin sub-micron fibers. In another embodiment, the sub-micron fibers are protein sub-micron fibers. In a further embodiment, the sub-micron fibers are combinations of cellulose sub-micron fibers, chitin sub-micron fibers and protein sub-micron fibers.

In some embodiments, the sub-micron fibers comprise oxidized cellulose. The term “oxidized” as used herein in reference to cellulose refers to cellulose wherein at least a portion of the hydroxyl groups in cellulose chains have been converted to carboxyl groups or a group comprising a carboxyl group (e.g., a carboxymethyl group).

The cellulose can be any suitable cellulose. In an embodiment, the cellulose is derived from a cellulose raw material that is wood-based (e.g., from a softwood tree such as pine, fir, larch, spruce, cypress or combinations thereof or a hardwood tree such as birch, poplar, basswood, eucalyptus, maple or combinations thereof) or from other plant sources such as straw, bagasse, bamboo, reed, jute, wheat, cotton, rice, flax or combinations thereof, or a non-plant based source such as bacteria and/or algae or combinations thereof. In another embodiment, the cellulose is derived from a cellulose source that is a suitable wood pulp such as but not limited to Northern bleached softwood kraft (NBSK), Southern bleached softwood kraft (SBSK) or combinations thereof. In a further embodiment, the cellulose source comprises, consists essentially of or consists of northern bleached softwood kraft (NBSK).

In an embodiment, at least a portion of the cellulose in the aerogel has a cellulose Iβ crystal structure. For example, in an embodiment, an X-ray powder diffraction (XRD) spectrum of the cellulose shows peaks at about 14.6°, about 15.4° and about 22.3° 2θ.

In an embodiment, a surface of the aerogel is modified with a hydrophobic surface modifying agent. The hydrophobic surface modifying agent can be any suitable hydrophobic surface modifying agent and can be readily selected by a person skilled in the art. In an embodiment, the hydrophobic surface modifying agent is a suitable silane, such as a silane of the Formula (I):

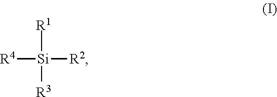

wherein R¹ is C₁₋₆alkyl and R², R³ and R⁴ are each independently chosen from C₁₋₆alkyl, hydroxy and a group that is hydrolysable under conditions to modify the surface, provided that at least one of R², R³ and R⁴ is a hydroxy or a group that is hydrolysable under conditions to modify the surface. A person skilled in the art would readily understand what groups are hydrolysable under particular conditions used to modify the surface and would be able to select a suitable silane and conditions accordingly. In an embodiment, R¹ is C₁₋₆ alkyl and R², R³ and R⁴ are each independently C₁₋₆alkoxy. In another embodiment, R¹ is methyl. In a further embodiment, R², R³ and R⁴ are each methoxy. In an embodiment, the hydrophobic surface modifying agent is methyltrimethoxysilane. It will be appreciated by a person skilled in the art that in embodiments wherein more than one of R², R³ and R⁴ are hydroxy and/or a group that is hydrolysable under conditions to modify the surface, the exact structure of the siloxane of the modified surface may vary. For example, an oxygen atom may be bonded to two silicon atoms, e.g., in a siloxane network, or to a silicon atom at one end and to a suitable atom in the remainder of the aerosol at the other, for example, a carbon atom, wherein the-Si—O—C-bond is formed from reaction with a surface hydroxyl of cellulose. Additionally, a person skilled in the art would understand that in such embodiments, optionally not all of the hydroxy groups and/or groups that are hydrolysable under conditions to modify the surface may participate in a condensation reaction such that the surface-modified aerosol may comprise variable amounts of Si—OH groups. In an embodiment, the weight concentration of Si in the surface modified aerogel is from about 10 wt % to about 20 wt %, about 12 wt % to about 18 wt % or about 15.4 wt %.

In an embodiment, the modified surface is superhydrophobic. In another embodiment, the water contact angle of the modified surface is greater than about 155°.

In an embodiment, the aerogel has isotropic superelasticity.

In an embodiment, density of the aerogel is from about 2 mg/m³ to about 22 mg/m³. In another embodiment, density of the aerogel is from about 2 mg/m³ to about 8 mg/m³, about 2 mg/m³ to about 6 mg/³, about 2 mg/m³ to about 2.4 mg/m³ or about 2.2 mg/m³. In another embodiment, density of the aerogel is from about 20 mg/m³ to about 20.4 mg/m³ or about 20.2 mg/m³.

In an embodiment, porosity of the aerogel is greater than about 98%. In another embodiment, porosity of the aerogel is from about 98.7% to about 99.8%, about 98.7% or greater, about 99.0% or greater, about 99.5% or greater or about 99.8%.

The present disclosure also includes a use of an aerogel of the present disclosure (including an aerogel prepared by a method of the present disclosure) for oil-water separation, as an absorbent, as a thermal insulator, as an acoustic insulator, as a drug carrier, as a tissue scaffold, as an infrared shield and/or in a sensor. In an embodiment, the aerogel is for use for oil-water separation. In another embodiment, the aerogel is for use as an absorbent. In a further embodiment, the aerogel is for use as a thermal insulator. In an embodiment, the aerogel is for use as an infrared shield. In another embodiment, the aerogel is for use in a sensor. In an embodiment, the absorbent is for use in environmental remediation such as oil contaminant removal. In another embodiment, the absorbent is for use in biomedical applications. In another embodiment, the thermal insulation is as an insulation layer in clothing. In another embodiment, the aerogel is for use as an insulation layer in a stress/strain sensor.

The following are non-limiting examples of the present disclosure:

### EXAMPLES

### Example 1: Multifunctional Superelastic Cellulose Nanofibrils Aerogel by Dual ice-Templating Assembly

### I. Experimental Section

(a) Materials and chemicals

Northern bleached softwood kraft pulp (NBSK) was provided by Canfor Corporation. 2,2,6,6-tetramethylpiperidine 1-oxyl (TEMPO, 98%) and sodium hypochlorite (NaClO, 11.9%, reagent grade) were purchased from Sigma-Aldrich. Sodium bromide (NaBr, 99%), Sodium hydroxide solution (NaOH, IN), methyltrimethoxysilane (MTMS, 97%), hexadecane, toluene, hexanes, and chloroform were purchased from Thermo Fisher Scientific. Canola oil, silicone oil and pump oil were purchased from local shops. All reagents were used without further purification.

### (b) Fabrication and Surface Modification of CNF Aerogel

Cellulose nanofibrils were obtained by TEMPO oxidization (2 mmol/g of NaClO) at pH 10.0, and then dialyzed against deionized water, and mechanical blending at 27,000 rpm (Vitamix 5200, 25 min) of NBSK. After blending, the CNF suspension was diluted to 0.2% and centrifuged at 2000 rpm for 10 min, and the precipitate was discarded. The sub-micron cellulose fibers were prepared by liquid nitrogen freezing (about 20-30 min) and freeze-drying (−50° C., 0.12 mBar, 48 h, Labconco Corporation, USA) of 0.05% CNF. After freeze-drying, the white, fluffy sub-micron CNF fibers were re-dispersed into aqueous suspension at varied concentrations of 0.2%, 0.4%, 0.6%, 0.8%, 1.0%, and 2.0% by vortex mixer. The suspended sub-micron CNF fibers were frozen at-20° C. for 6 h and then freeze-dried (−50° C., 0.12 mBar, 48 h), yielding sub-micron CNF aerogel. The derived CNF aerogels were hydrophobized by chemical vapor deposition of MTMS in a closed container containing 1 mL MTMS and 0.5 mL water at 80° C. for 6 hours.

### (c) Characterization

Brunauer-Emmett-Teller (BET) surface area measurement: The specific surface area and pore characteristics of the sub-micron CNF fibers and 0.2 wt % CNF aerogel were determined by nitrogen adsorption-desorption method using a Quadrasorb SI (Quantachrome Instruments, US). Pore size distributions were determined from the adsorption curve of the isotherm using the density functional theory (DFT) method.

Scanning Electron Microscope (SEM): The aerogels were cut using a sharp razor along the cross section to expose the internal structure. The microstructures and morphologies of the aerogel were characterized by field-emission scanning electron microscope (FE-SEM, Sigma 500).

Fourier transform infrared spectroscopy (FTIR): The chemical structures of unmodified and MTMS coated CNF aerogel were analyzed by FTIR spectroscopy (Varian 3100, Varian, Inc., Palo Alto, CA) from 4000 to 600 cm⁻¹ with a spectral resolution of 4 cm⁻¹ and 80 scans.

Thermal conductivity and infrared imaging: Thermal conductivity and diffusivity of the aerogels were determined by the transient plane source (TPS) method using a thermal conductivity analyser (TPS 2500 S) under ambient conditions. The infrared (IR) image and upper surface temperature of aerogel placed either on an 80° C. hot plate or −17° C. ice cube were recorded using an infrared thermal imager (Rohs HT-19).

Mechanical testing: The mechanical properties of the aerogels were measured by a compression test. Cylindrical aerogels (diameter: 21 mm, height: 23 mm) were compressed using an Instron 3345 Materials Testing System with a 2 kN load cell at 1 mm/min displacement rate until reaching 60% compressive strain, and the Young's modulus and yield strength were calculated from the initial linear elastic region of the stress-strain curve. Due to the high hydrophobicity, the aerogel has a very low moisture content of 1.16±0.11% as determined by heating the sample in a 105° C. oven for 24 h.

Water contact angle: The water contact angles of hydrophobic aerogels were measured with a DataPhysics PSL 250 contact angle analyzer (Future Digital Scientific Corp., GmbH, Germany). 4 μL deionized water was dropped on the aerogel surface using a micro syringe. The contact angle was measured every 1 s for 1 min.

### II. Results and Discussion

(a) Dual ice-Templating Assembly of Superelastic CNF Aerogel

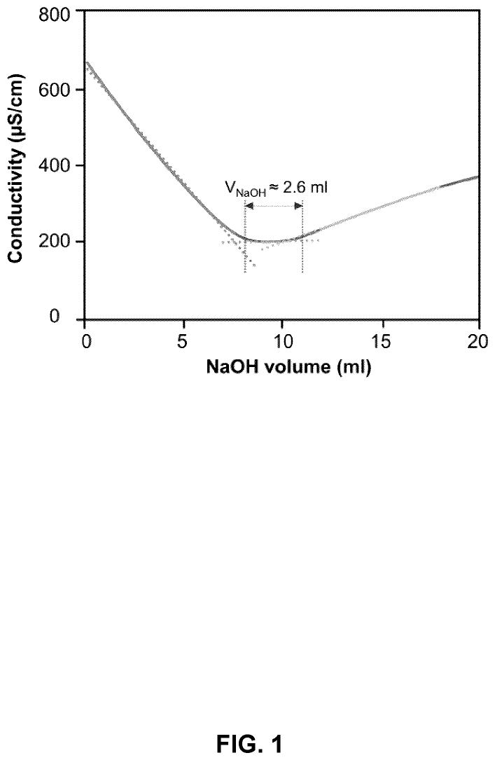

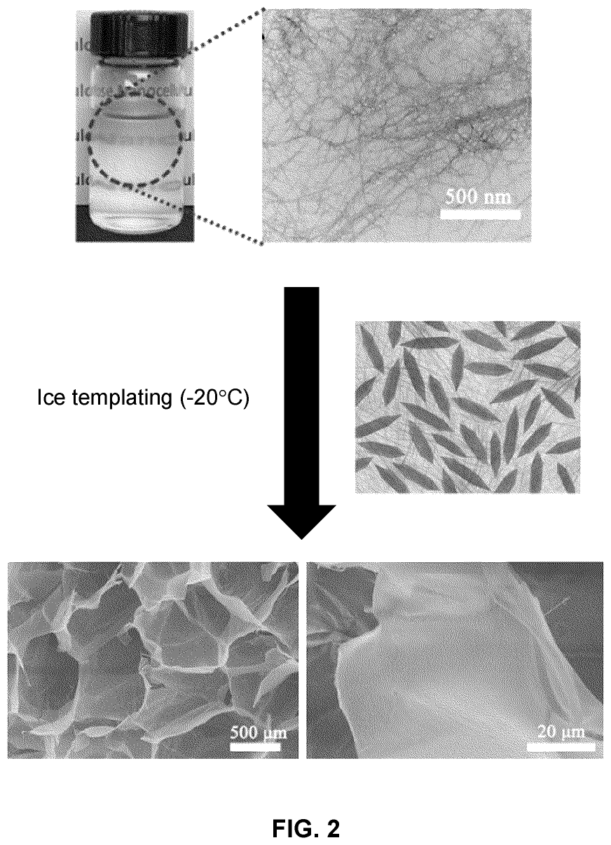

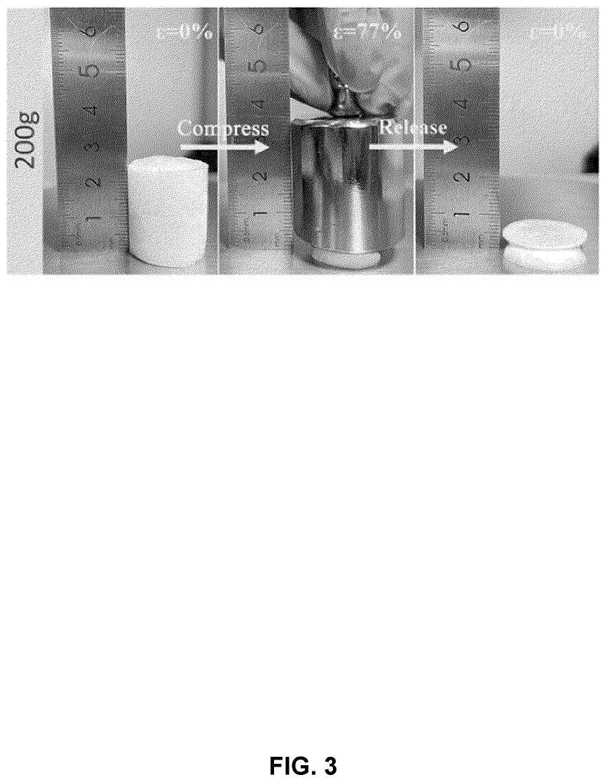

Due to the presence of abundant surface polar carboxyl (0.385 mmol/g, FIG. 1) and hydroxyl groups, 2,2,6,6-tetramethylpiperidine 1-oxyl (TEMPO)-oxidized cellulose nanofibrils (CNFs) can assemble into various morphologies (such as fibers, ribbons, and films) via ice-templating methods (Jiang & Hsieh, 2014a; Jiang & Hsieh, 2014b). Such assembled morphologies can be facilely controlled by different freezing temperatures, developing a sub-micron fibrous morphology when exposed to extremely low temperature (such as using liquid nitrogen of −196° C.) due to the inhibition of the growth of large ice crystals. Conventionally, CNF aerogel can be fabricated by directly freezing its aqueous suspension at −20° C., yielding a honeycomb structure with a few hundred micrometer large pores, due to the growth of large ice crystals under low freezing rate at the relative high temperature (FIG. 2). During such process, the CNF can extensively assemble into dense films with low elasticity. The as-prepared CNF aerogel can be easily compressed by a 200 g weight to 77% strain, forming a thin disk that cannot recover to the original shape (FIG. 3). Such non-elastic performance of the CNF aerogel has greatly restricted its applications in areas that shape-recovery is essential, such as insulation layers in clothing and stress/strain sensors.

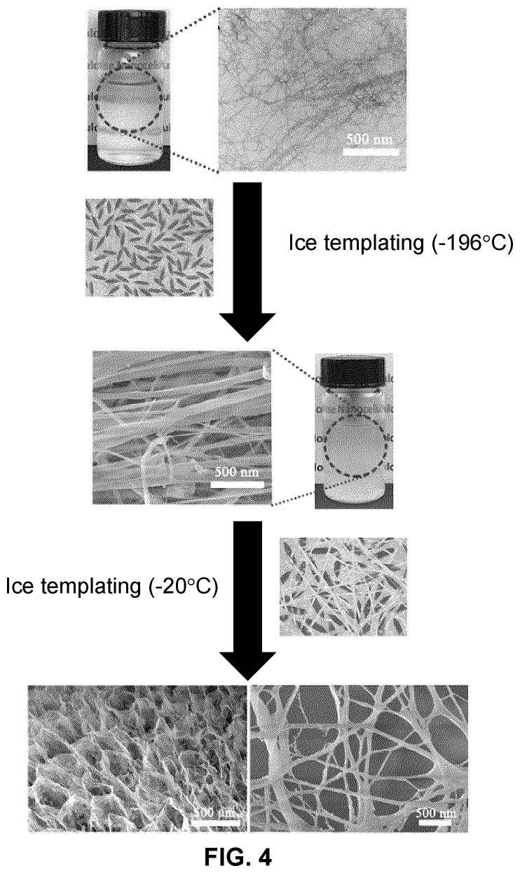

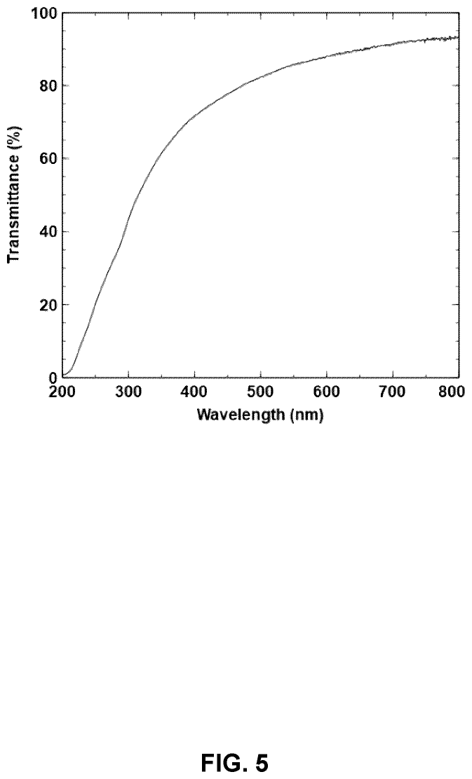

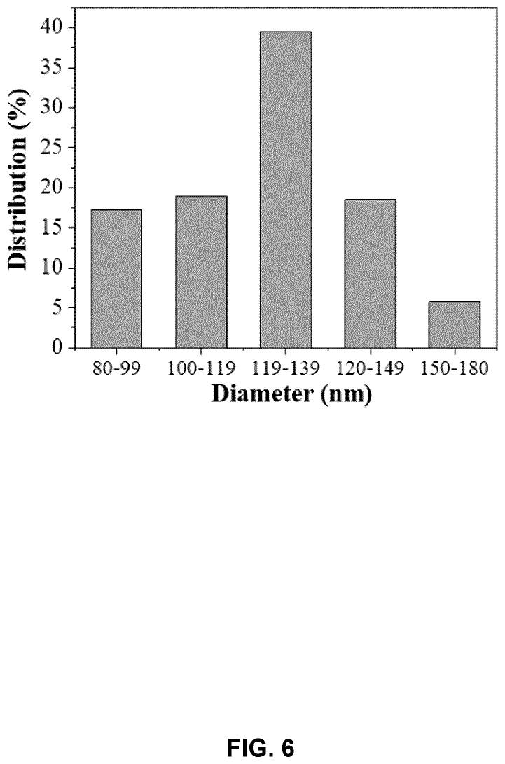

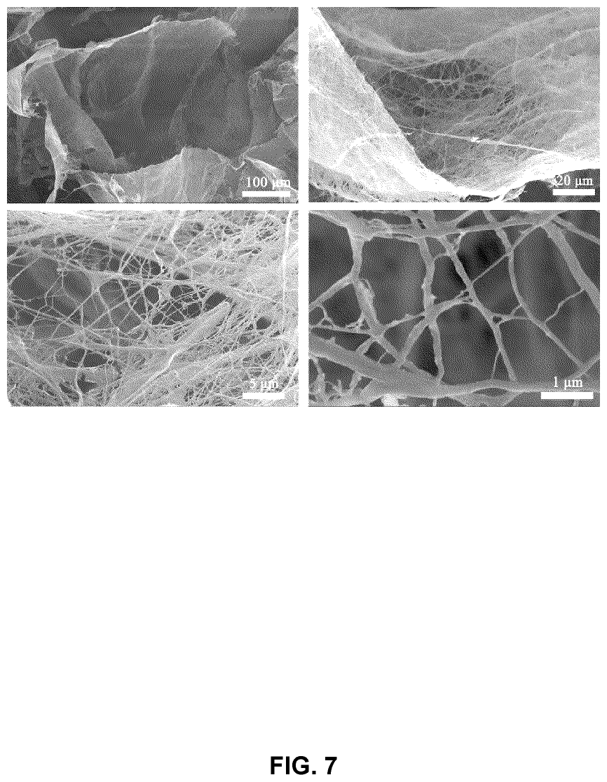

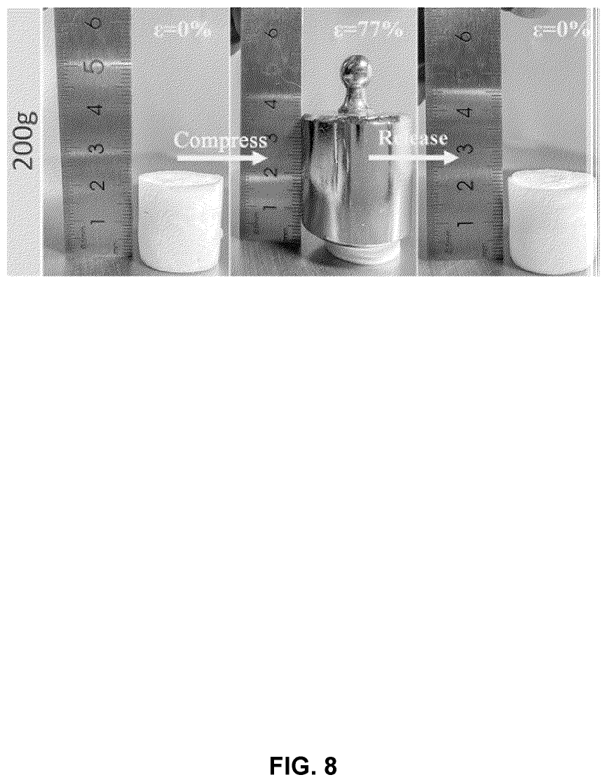

In this work, inspired from the dramatically different assembled morphologies impacted by changing the freezing conditions, a superelastic CNF aerogel was developed following two steps, including (i) assembling TEMPO-oxidized CNFs (3-5 nm wide and 500-1000 nm long) at −196° C. into continuous sub-micron fibers (100-200 nm wide) with improved elasticity: and (ii) constructing hierarchical lamellar fibrous structure for enhanced bulk elasticity by freezing the pre-assembled sub-micron fibers at −20° C. (FIG. 4). As both processes are realized by an ice-templating technique, such a strategy can be referred to as dual ice-templating assembly (DITA). The CNF suspension showed high transparency (90% at 650 nm wavelength, FIG. 5) due to the nanoscale dimension as revealed from TEM imaging (top images in FIG. 2 and FIG. 4). Freezing a dilute CNF suspension (0.05 wt %) at −196° C. followed by freeze-drying can turn the nanoscale CNF into sub-micron fibers with an average diameter of 131±40 nm (middle image in FIG. 4; FIG. 6). The sub-micron fibers can be re-dispersed into water at various concentrations and then frozen at −20° C. and freeze-dried to form a superelastic aerogel. SEM images of the CNF aerogel (from 0.2 wt % sub-micron fibers) from the DITA process showed a honeycomb structure due to the large ice crystals formed at −20° C., and the cell walls are composed of interconnected sub-micron fibers with similar diameters as compared to the original sub-micron fibers (bottom image in FIG. 4: FIG. 7). This indicates that the sub-micron fibers do not assemble with each other during the second ice-templating stage, while not wishing to be limited by theory, possibly due to the reduced surface area of the sub-micron fibers and lack of tendency for inter-fiber interactions. In contrast to the conventional CNF aerogel, the CNF aerogel from the DITA process shows excellent elastic behavior along the longitudinal direction that can recover to its original dimension after removing the 200 g weight (FIG. 8). Such good elasticity can be ascribed to the presence of interconnected superelastic sub-micron fibers.

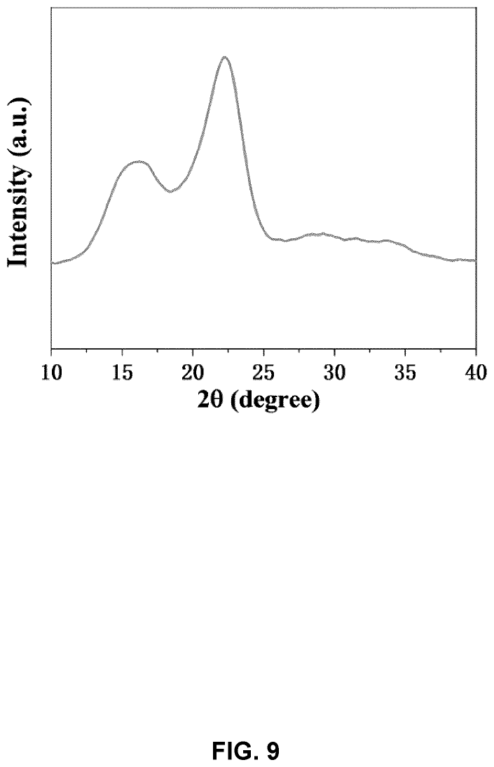

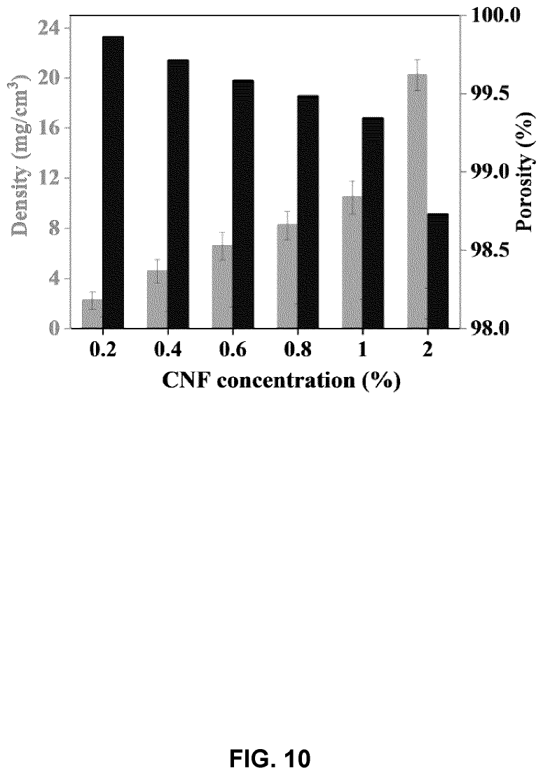

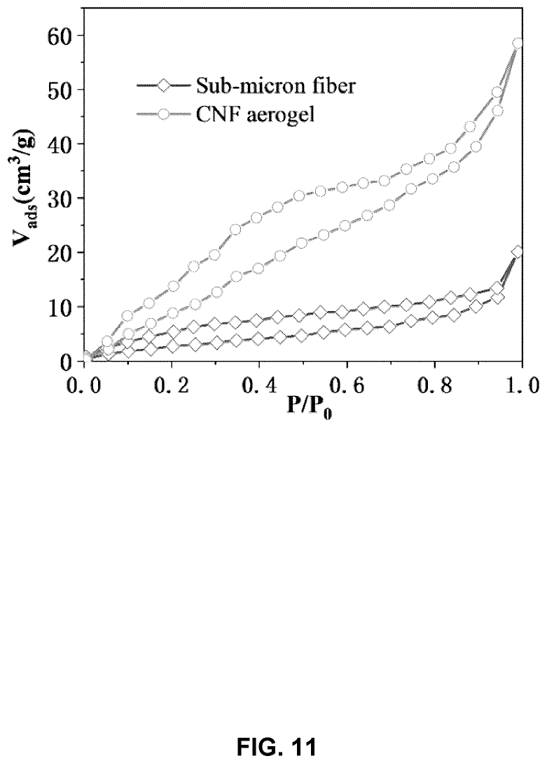

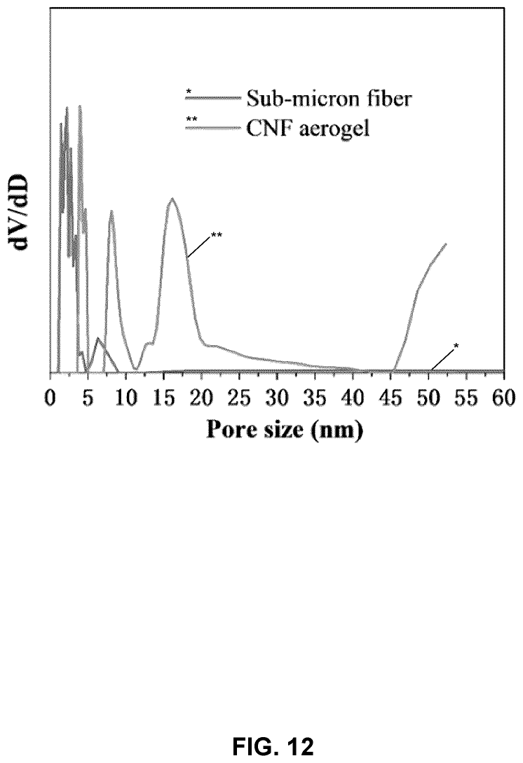

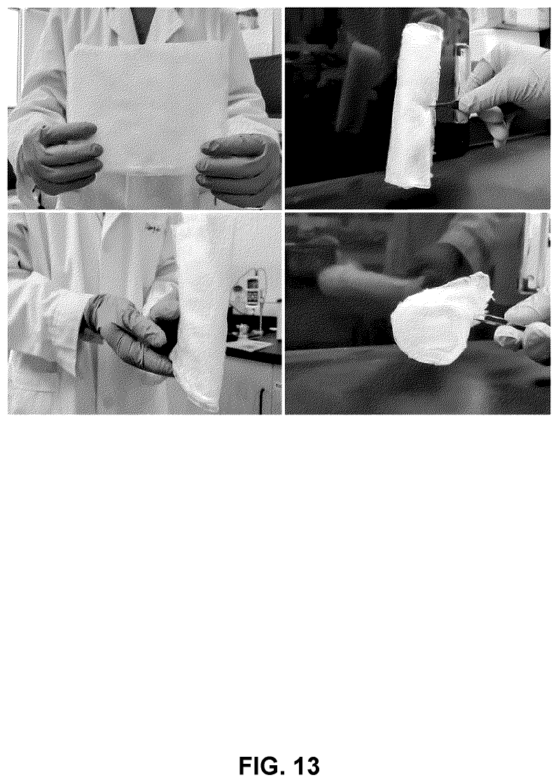

The assembled sub-micron CNF fibers preserved cellulose Iβ crystal structure according to XRD spectrum (FIG. 9), showing characteristics peaks at 14.6, 15.4, and 22.3° that correspond to the 11̅0, 110, and 200 crystallographic planes of the monoclinic cellulose Iβ allomorph. The crystallinity index was determined as 42.5%. CNF aerogel with various densities (2.2-20.2 mg/m3) and porosities (99.8-98.7%) can be assembled by tuning the initial sub-micron CNF fiber concentrations from 0.2 to 2 wt % (FIG. 10). Due to the entangled structure, dispersing sub-micron CNF fibers at a concentration greater than 2 wt % is difficult to achieve good homogeneity. N2 adsorption-desorption curves for the sub-micron CNF fibers and aerogel showed a type IV isotherm with a specific surface area and pore volume of 11.4 m²/g, 0.058 cm³/g and 34.1 m²/g, 0.133 m³/g, respectively (FIG. 11). While not wishing to be limited by theory, the increased specific surface area and pore volume after the second ice templating assembly may originate from the formation of an aerogel with more mesoporous, stack pores and macropores. This can be corroborated from the additional peaks in the pore size distribution curve of the CNF aerogel (FIG. 12). The sub-micron CNF aerogel displayed a H3-type hysteresis loop in the relative pressure (P/Po) range of 0.3-0.7, which indicates mesoporous structure. In addition, the strong uptake around 0.9-1.0 suggested a macroporous structure, which is expected to be formed during ice-templating at relatively higher freezing temperatures. A large piece of CNF aerogel mat can also be produced, showing excellent flexibility and foldability that can be rolled and folded without breaking (FIG. 13).

### (b) Hydrophobic Surface Modification of Superelastic CNF Aerogel

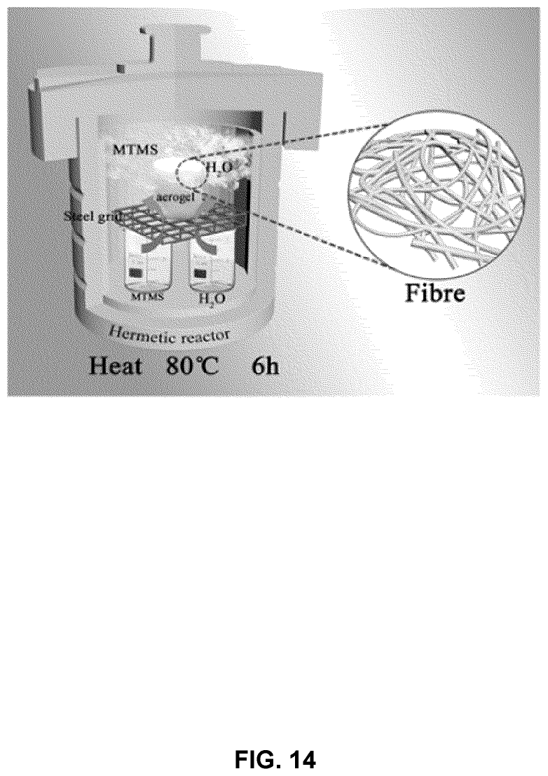

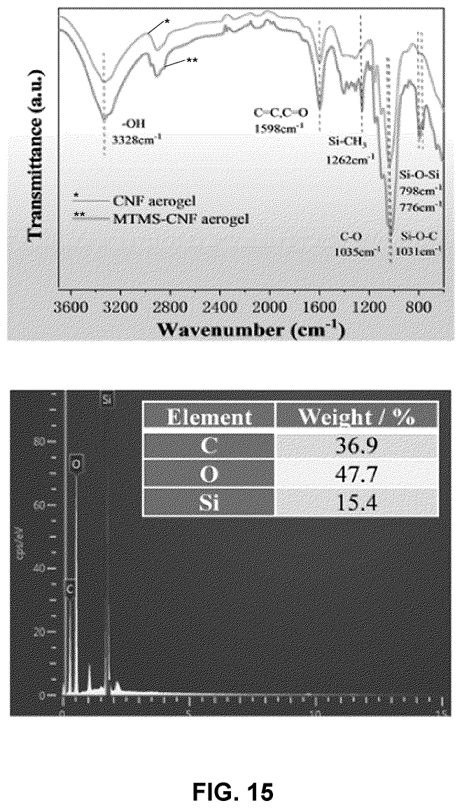

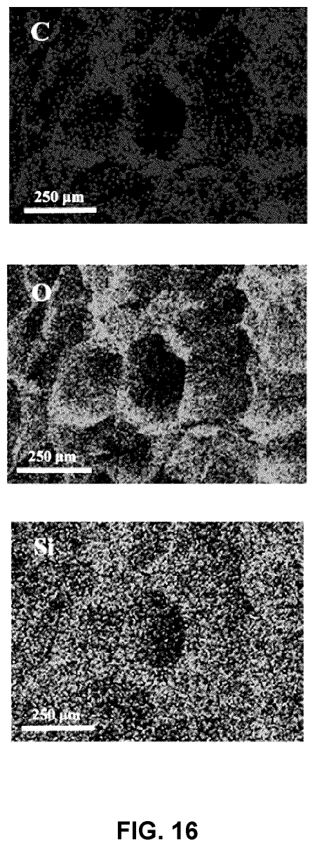

The CNF aerogel is hydrophilic and can be easily dispersed into water under gentle stirring. To improve its aqueous stability and hydrophobicity, CNF aerogel was facilely hydrophobized by chemical vapor deposition of methyltrimethoxysilane (MTMS) at 80° C. under vacuum for 6 h (FIG. 14). The successful silane modification can be confirmed from FTIR (FIG. 15; top) and EDS spectra (FIG. 15; bottom). The characteristic peaks at 3328 cm⁻¹, 1598 cm⁻¹ and 1035 cm⁻¹ indicate the O—H stretching vibration, C═O stretching vibration (in the form of sodium carboxylate) and C—O stretching vibration (Jiang et al., 2016), all appearing in the aerogel before and after the silane modification. Other than the typical cellulose peaks observed in unmodified CNF aerogel, the silane modified MTMS-CNF aerogel shows distinct peaks at 1262 cm⁻¹, 776 cm⁻¹, 798 cm⁻¹ for Si—CH₃, and Si—O—Si, indicating the formation of siloxane network. The C—O stretching peak of the CNF aerogel can be observed as 1035 cm⁻¹. After silane modification, the peak shifted to 1031 cm⁻¹ due to the overlapping with the stretching vibration of Si—O—C peak, indicating that the MTMS can also react with the surface hydroxyls of CNF to form covalent bonds (Baatti et al., 2019). C, O, and Si atoms are clearly observed in the EDS mapping images (FIG. 16), and the weight concentration of Si was determined as 15.4%.

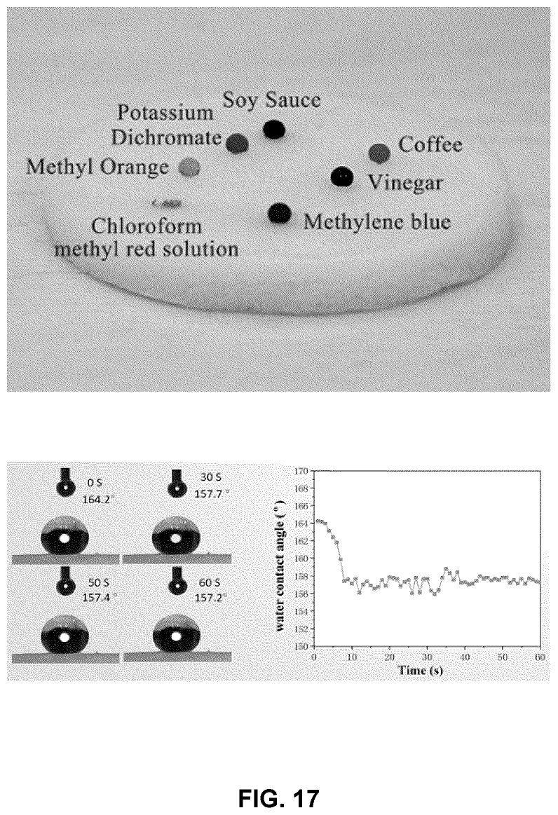

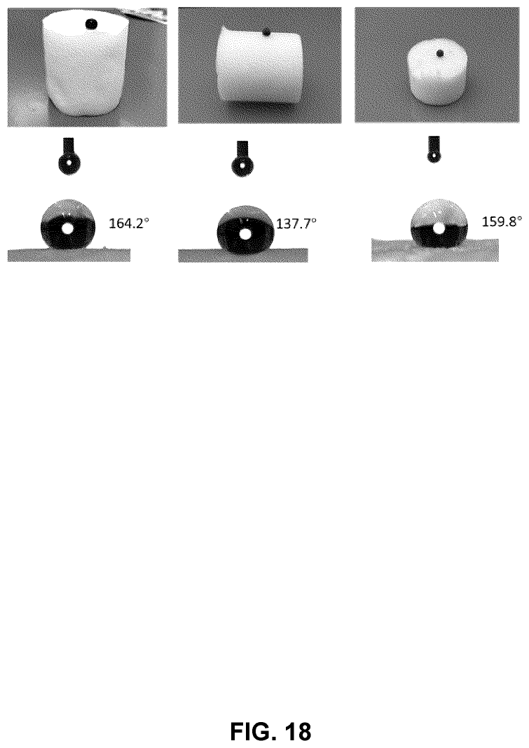

The MTMS-CNF aerogel showed high hydrophobicity due to the presence of non-polar methyl groups, as demonstrated from the spherical beads of different dyed water and drink/sauces on top of the aerogel (FIG. 17; top). However, non-polar organic solvents (such as chloroform) can still be absorbed by the hydrophobic aerogel. The top surface of the MTMS-CNF aerogel showed an initially high water contact angle of 164.2°, which then gradually reduced to 157.7° after 7 seconds and then stabilized (FIG. 17; bottom). Other than the top surface, the MTMS-CNF aerogel also showed hydrophobicity and a high contact angle on the side surface and even the interior exposed from cutting (FIG. 18), indicating the chemical deposition of MTMS can also penetrate into the aerogel. The 164° water contact angle being over 150° can be considered as superhydrophobic (Law, 2014), and is significantly higher than previously reported cellulose-based aerogel (135-154°, Table 1: Cervin et al., 2012; H. M. Zhang et al., 2021; Wang et al., 2015; Wang et al., 2020).

TABLE 1
Comparison of contact angle of cellulose-based aerogels.

| | | Contact |
|---|---|---|
| Samples | Hydrophobic modification | angle (°) |
| Carboxymethylated | Octyltrichlorosilane vapor | 150 |
| CNF | deposition | |
| Recycled cellulose | Methyltrimethoxysilane vapor | 153.5 |
| fibers | deposition | |
| Cellulose nanofibrils | Styrene-acrylic modification | 149 |
| Microfibrillated cellulose | Methyltrimethoxysilane vapor | 154 |
| fibers | deposition | |
| Cellulose nanofibers | Fluorinated finishing agent | 135 |
| Kapok fibers/ | Vinyltrimethoxysilane | 140 |
| microfibrillated cellulose | modification | |
| DITA CNF | Methyltrimethoxysilane vapor | 164.2 |
| (described herein) | deposition | |

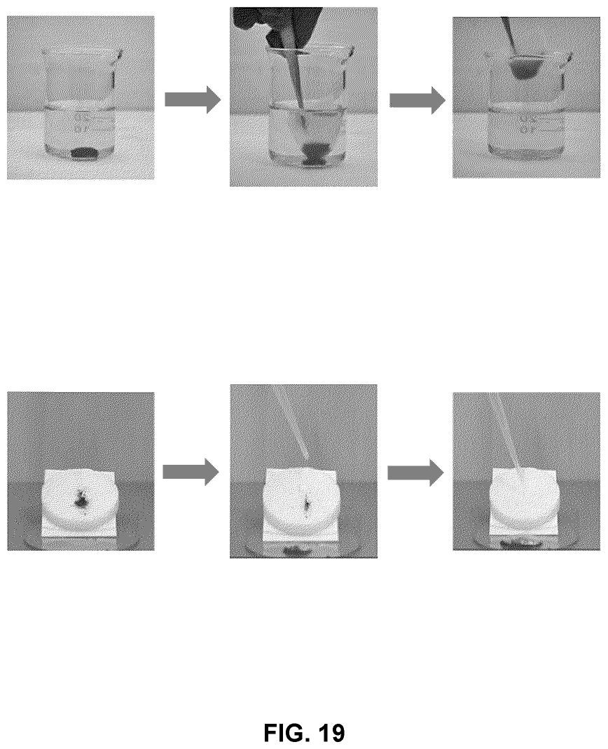

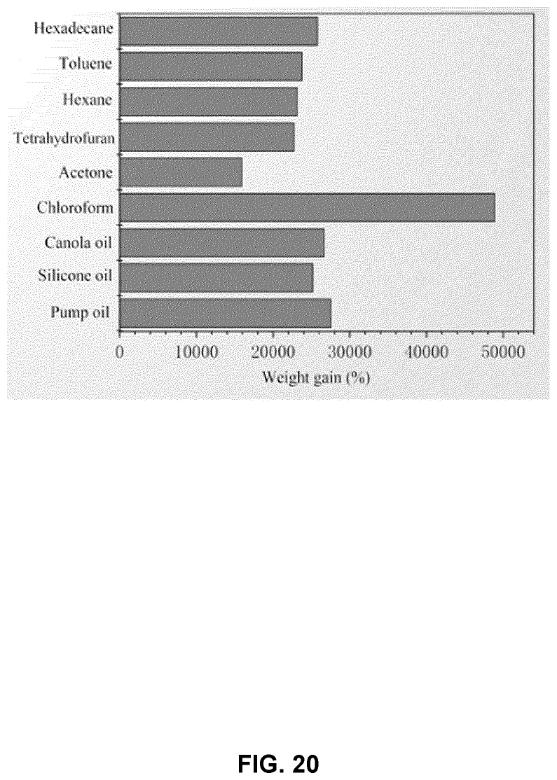

Due to its selective absorption toward non-polar liquids after hydrophobization, the MTMS-CNF aerogel can be used, for example, for oil-water separation by selectively removing oil (chloroform) from the bottom of water (FIG. 19). In addition, the superhydrophobic MTMS-CNF aerogel demonstrated self-cleaning performance as the dirt on top of the aerogel can be easily washed off with water (FIG. 20).

In order to further investigate the absorption properties of the hydrophobic aerogel, various kinds of oil (e.g. pump oil, silicone oil, and canola oil) and organic solvents (e.g. hexadecane, toluene, hexane, tetrahydrofuran, acetone, and chloroform) were used as absorbates, which are the main pollutants in industry and daily life (FIG. 20). The weight gain (wt %) is defined as the weight ratio of the absorbate to the dried aerogel. The MTMS-CNF aerogel showed very high absorption capacity for all tested oils and organic solvents, showing high weight gains of 25765%, 23416%, and 48886% for pump oil, canola oil and chloroform, respectively. The absorption capacity of MTMS-CNF aerogel towards chloroform reached 489 times its own weight, which is significantly higher than the conventional CNF aerogel with similar density (375 g/g) (Jiang & Hsieh, 2014a). The increased absorption capacity over conventional CNF aerogel can be ascribed to the increased solvent accessibility due to the interconnected open cell wall structure. The conventional CNF aerogel had dense film-like cell wall structures that can restrict the liquid accessibility. In addition, the 489 g/g absorption capacity towards chloroform is significantly higher than other superabsorbent aerogels, including polyimide nanofiber/MXene aerogel (135 g/g toward CC14) (Liu et al., 2021), chitosan aerogel (117 g/g: Cao et al., 2021), silylated cellulose nanofibrils aerogel (205 g/g: Laitinen et al., 2017), bacterial cellulose-polymethylsilsesquioxane aerogel (203 g/g: J. Y. Zhang et al., 2021), carbon nanofibers aerogel (290 g/g: Wu et al., 2013), and carbon nanotube sponge (180 g/g: Gui et al., 2010), and is close to the graphene framework (480 g/g: Zhao et al., 2012). In general, the MTMS-CNF aerogel demonstrates very high organic absorption of up to 489 times of its own weight, suggesting that it can serve as an effective absorbent for environmental remediation.

### (c) Mechanical Properties and Superelastic Performance

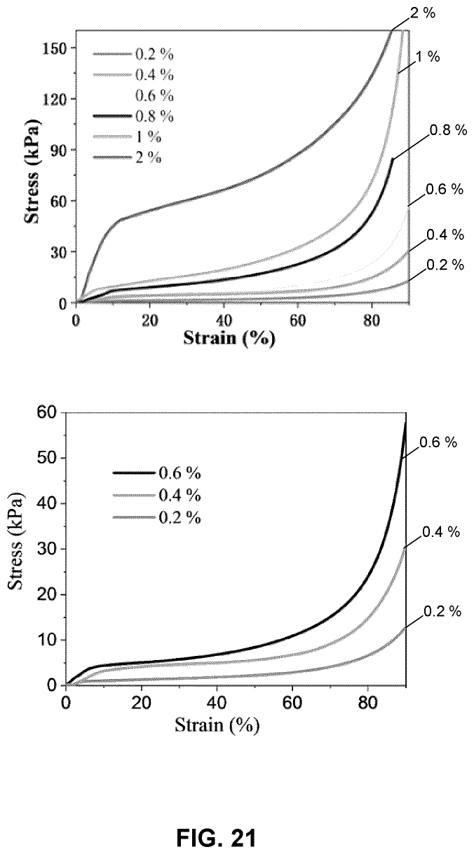

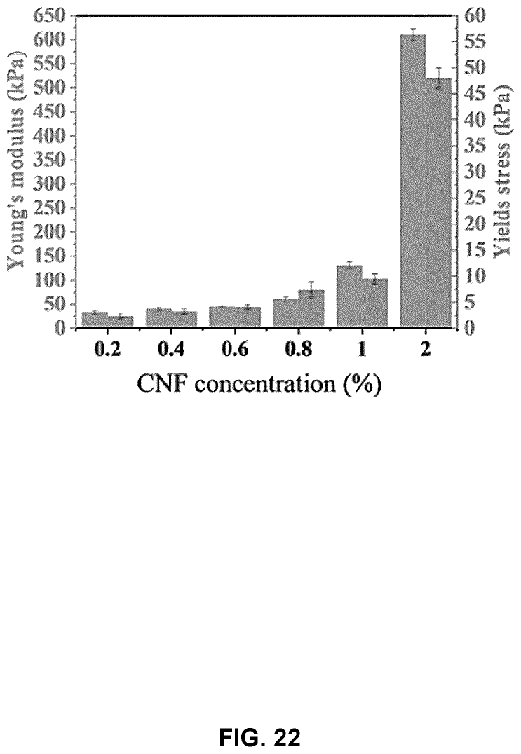

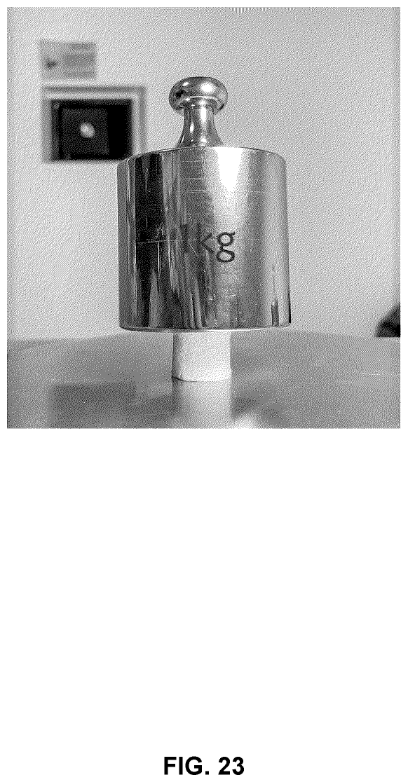

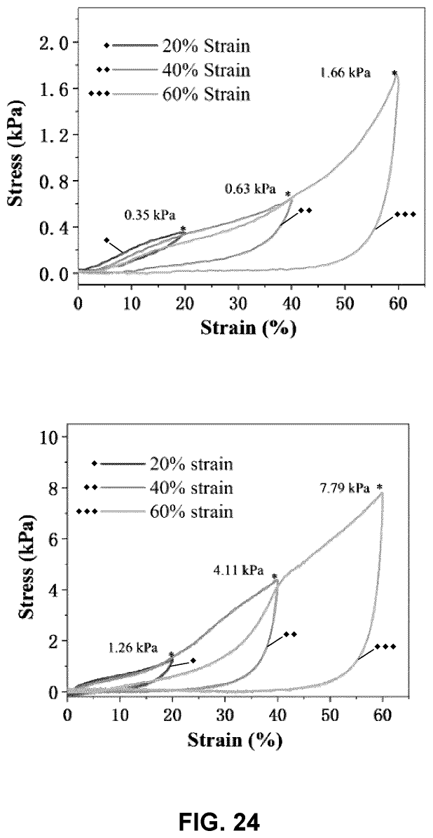

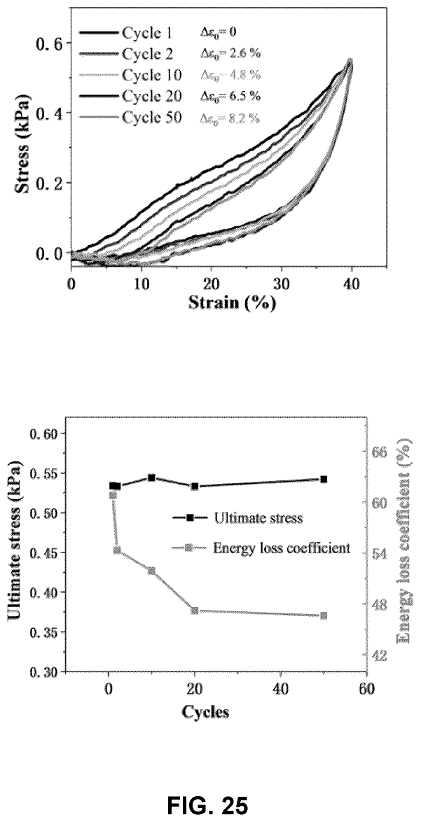

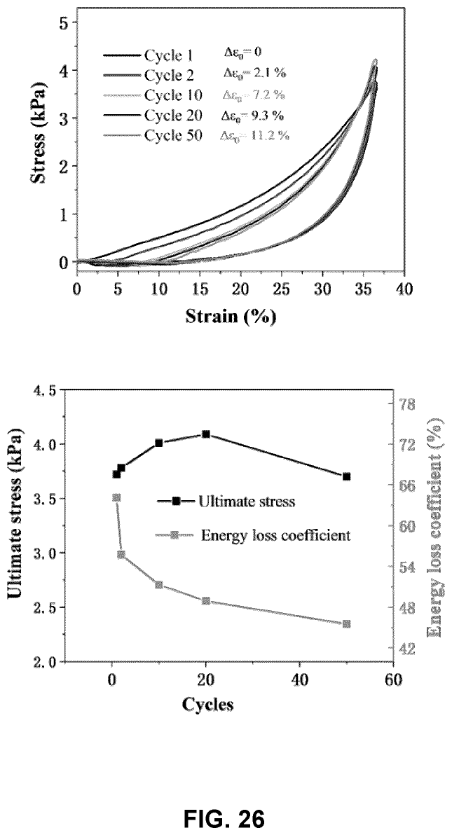

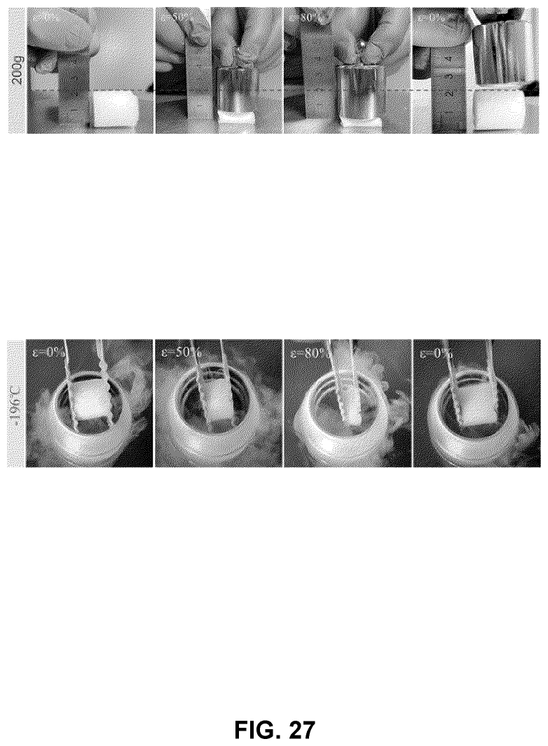

Compressive mechanical performance of the aerogel is critical in determining its durability and suitability for either oil/water separation or thermal insulation applications. Compressive stress-strain (σ-ε) curves of the MTMS-CNF aerogel with different initial concentrations (corresponding to different densities as shown in FIG. 10) showed increased strength with increasing aerogel densities. All aerogels can be compressed to over 80% strain, indicating excellent flexibility and non-brittleness, which is in sharp contrast to conventional inorganic aerogels (such as SiO₂) that shatter upon gentle touch. All stress-strain curves showed three stages that are characteristic to foam-like structures, including a linear elastic region at low strain values (less than 10%) prior to the yield point, a plastic region with a relatively flat plateau curve at medium strain values (from 10-70% strain), and the last densification stage with a sharply increased stress at high strain values (over 70% strain) (FIG. 21). With increasing aerogel density, a more well-defined yield point can be observed, indicating the transition from an elastic aerogel to a more rigid foam at a higher density. Both Young's modulus and the yield stress gradually increased with increasing initial concentration up to 1%, and then dramatically increased to 610 and 48 kPa at 2%, respectively (FIG. 22). The assembled aerogel from 2% CNF demonstrated rigid behavior that can support over 5376 times its own weight (FIG. 23). The dramatically increased mechanical performance at 2% CNF concentration can be ascribed to the increased density and greater interconnection of the adjacent fibers. Therefore, by using this dual ice-templating assembly technique, both superelastic and rigid/strong aerogels can be obtained depending on the initial CNF concentration. Cyclic stress-strain curves of the CNF aerogel assembled from CNF suspensions with initial concentrations of 0.2% and 0.6% revealed elastic behavior even at a high strain of 60% (FIG. 24). Cyclic compressive stress-strain curves of 0.2% CNF aerogel showed good elastic behavior over 50 cycles (FIG. 25; top). The aerogel can almost recover to its original dimension after compression, showing as low as 8.2% unrecoverable strain after 50 cycles. The ultimate stress maintained at 0.542 kPa, which is close to the first cycle, while not wishing to be limited by theory, suggesting the macroscopic structure is well-maintained (FIG. 25; bottom). High energy loss (60.8% energy loss coefficient) is observed from the first cycle, which can be ascribed to the buckling and breaking of the interconnected sub-micron fibers within the cell walls. After 20 cycles the energy loss coefficient stabilized at 47.2%, indicating minimal structural changes during further cyclic compression testing. Similar cyclic compressive stress-strain curves can also be observed for the 0.6% CNF aerogel, with approximately 11.2% unrecovered strain after 50 cycles (FIG. 26). The 0.2% CNF aerogel demonstrated isotropic superelastic behavior, showing instantaneous shape recovery from large >80% compression strains along both longitudinal (FIG. 8) and cross-sectional directions FIG. 27; upper images). More surprisingly, the aerogel demonstrated superelastic behavior even at an extremely low temperature, such as −196° C. in liquid nitrogen (FIG. 27; lower images). Such excellent superelastic behaviour under extreme conditions is desired for its application as a thermal insulation layer in cold environment, without losing its flexibility.

### (d) Thermal Insulating Performance of CNF Aerogel

As aerogel is widely used for thermal insulation due to its low density and highly porous internal structure, the thermal insulating performance of the aerogel was further evaluated. The 0.2% CNF aerogel showed a very low thermal conductivity of 0.023 W/(m·K), which is even lower than that of air (0.025 W/(m·K); Tang et al., 2008). The low thermal conductivity is close to other nanocellulose-based aerogels (Table 2).

TABLE 2
Comparison of thermal conductivity of cellulose-based aerogels.

| | | Thermal |
|---|---|---|
| | Density | conductivity |
| Samples | (mg/cm³) | (W/(m · K)) |
| Liquid-crystalline nanocellulose aerogel | 17 | 0.018 |
| Regenerated cellulose aerogel | 20-30 | 0.03 |
| Polymethylsilsesquioxane-cellulose | 14.2 | 0.0153 |
| nanofiber aerogel | | |
| Nanofibrillated cellulose aerogel | 8-20 | 0.026-0.039 |
| CNF aerogel fibers | 200 | 0.07 |
| Bleached cellulose fibers aerogel | 15-160 | 0.023-0.028 |
| Regenerated cellulose aerogel | 9-137 | 0.04-0.075 |
| Pickering emulsion CNF aerogel | 11.4-22 | 0.0155-0.0214 |
| DITA CNF aerogel (described herein) | 2.2 | 0.023 |

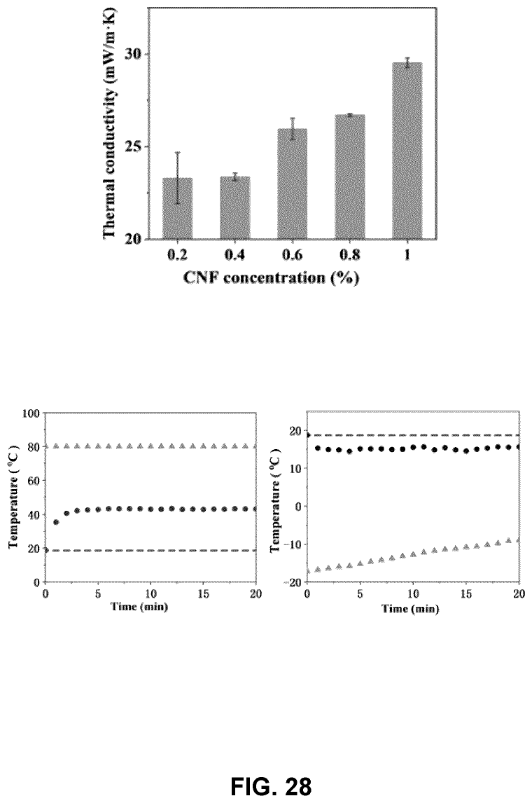

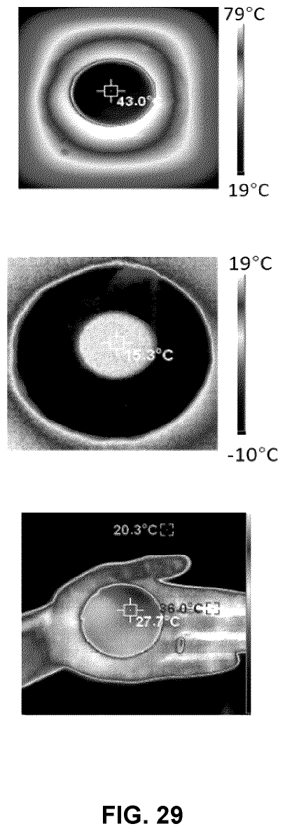

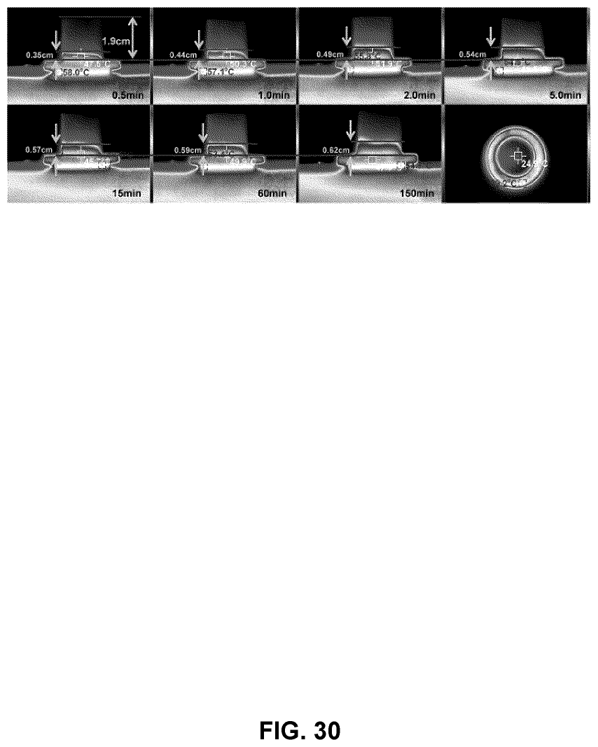

The thermal conductivity of the aerogel increased with an increase in the initial CNF concentration, reaching to 0.029 W/(m·K) for the 1% CNF aerogel (FIG. 28; upper plot). A higher thermal conductivity at increased density is expected as the heat conduction through solid networks will increase. The effects of 0.2% CNF aerogel in inhibiting heat transfer is further demonstrated by placing a thin piece of aerogel on either a hot (hot plate at 80° C.) or cold surfaces (ice cube at −17° C.). The 8 mm aerogel can effectively block heat/cold transfer, showing a 37° C. and 25° C. temperature difference between the opposite surfaces on the hot and cold surfaces, respectively (FIG. 28; lower plots and FIG. 29; upper and middle images). The progressive heat transfer through the aerogel was visualized using an IR camera on top of a hot surface (FIG. 30). Heat transfer through the bottom surface can be observed during the first 15 min, with a penetration depth of 0.57 cm. The heat penetration is reduced further with increased time, and the penetration depth only reaches to 0.62 cm after 150 min. The top surface of the aerogel remained at 24.9° C., suggesting the heat is effectively blocked by the aerogel. Due to its low thermal conductivity and porous structure, the CNF aerogel can also be used to block IR transmission, yielding IR shielding performance (FIG. 29; bottom image).

A dual ice-templating assembly (DITA) technique was developed to assemble cellulose nanofibrils into a superelastic aerogel. By firstly assembling the ultrathin (3-5 nm wide) CNFs into continuous sub-micron (100-200 nm in diameter) fibers, assembly of the CNFs into a film-like structure will be inhibited due to the significantly reduced surface area. The interconnected sub-micron fibers network provides excellent elastic behavior, resulting in a shape recovery CNF aerogel under high strain. Such superelasticity can even be well maintained under an extremely cold environments such as liquid nitrogen, making it ideal for applications in extreme temperatures. The CNF aerogel was further hydrophobized using MTMS via a chemical vapor deposition method. The hydrophobic CNF aerogel showed a high water contact angle of 164°, and can be used, for example, to selectively remove oil from water and benefit self-cleaning by removing the contaminants from the aerogel surface. Due to its low density, the CNF aerogel showed a high absorption capacity towards various oil and organic solvents, ranging from 234-489 times its own weight, significantly higher than most currently reported aerogels. The CNF aerogel showed very low thermal conductivity, as low as 0.023 W/(m·K), and demonstrated excellent thermal insulating and infrared shielding performance. Therefore, this work presents a novel technology in assembling cellulose nanofibrils into superelastic and multifunctional aerogels, which can be used in many applications such as superabsorbent materials, thermal insulation, and oil/water separation. The proposed strategy is also expected to be applied to other bio-based nanomaterials to produce such superelastic aerogels.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE DESCRIPTION

A. Baatti, F. Erchiqui, P. Bebin, F. Godard, D. Bussieres, Canadian Journal of Chemical Engineering 2019, 97, 2050.
J. Cai, S. Kimura, M. Wada, S. Kuga, L. Zhang, ChemSusChem 2008, 1, 149.
M. Cao, S. L. Li, J. B. Cheng, A. N. Zhang, Y. Z. Wang, H. B. Zhao, Journal of Hazardous Materials 2021, 403.
N. T. Cervin, C. Aulin, P. T. Larsson, L. Wagberg, Cellulose 2012, 19, 401.
Y. Chen, D. B. Fan, S. Y. Lyu, G. Y. Li, F. Jiang, S. Q. Wang, ACS Sustainable Chemistry & Engineering 2019, 7, 1381.
Y. M. Chen, L. Zhang, Y. Yang, B. Pang, W. H. Xu, G. G. Duan, S. H. Jiang, K. Zhang, Advanced Materials 2021, 33.
W. M. Chen, S. Chen, Y. Morsi, H. El-Hamshary, M. El-Newehy, C. Y. Fan, X. M. Mo, ACS Appl. Mater. Interfaces 2016, 8, 24415.
Z. H. Chen, H. Zhuo, Y. J. Hu, H. H. Lai, L. X. Liu, L. X. Zhong, X. W. Peng, Adv. Funct. Mater. 2020, 30, 1910292.
Cho, M. Karaaslan, H. Wang, S. Renneckar, J. Mater. Chem. A 2018, 6, 20973.
Dou, X. X. Zhang, H. R. Shan, X. T. Cheng, Y. Si, J. Y. Yu, B. Ding, Adv. Funct. Mater. 2020, 30, 2005928.
E. S. Ferreira, C. A. Rezende, E. D. Cranston, Green Chemistry 2021, 23, 3542.
H. Guan, Z. Y. Cheng, X. Q. Wang, ACS Nano 2018, 12, 10365.
X. C. Gui, J. Q. Wei, K. L. Wang, A. Y. Cao, H. W. Zhu, Y. Jia, Q. K. Shu, D. H. Wu, Advanced Materials 2010, 22, 617.
J. K. Huang, H. B. Wang, B. Q. Liang, U. J. Etim, Y. X. Liu, Y. P. Li, Z. F. Yan, Chem. Eng. J. 2019, 364, 28.
C. Jia, L. Li, Y. Liu, B. Fang, H. Ding, J. N. Song, Y. B. Liu, K. J. Xiang, S. Lin, Z. W. Li, W. J. Si, B. Li, X. Sheng, D. Z. Wang, X. D. Wei, H. Wu, Nat. Commun. 2020, 11, 3732.
A. Isogai, T. Hänninen, S. Fujisawa, T. Saito, Progress in Polymer Science 2018, 86, 122-148.
F. Jiang, Y. L. Hsieh, J. Mater. Chem. A 2014a, 2, 6337.
F. Jiang, Y.-L. Hsieh, ACS Applied Materials & Interfaces 2014b, 6, 20075.
F. Jiang, Y. L. Hsieh, ACS Sustainable Chemistry & Engineering 2016, 4, 1041.
F. Jiang, H. Liu, Y. Li, Y. D. Kuang, X. Xu, C. J. Chen, H. Huang, C. Jia, X. P. Zhao, E. Hitz, Y. B. Zhou, R. G. Yang, L. F. Cui, L. B. Hu, ACS Appl. Mater. Interfaces 2018, 10, 1104.
H. Jin, Y. Nishiyama, M. Wada, S. Kuga, Colloids and Surfaces A: Physicochemical and Engineering Aspects 2004, 240, 63.
K. Y. Law, Journal of Physical Chemistry Letters 2014, 5, 686.
O. Laitinen, T. Suopajarvi, M. Osterberg, H. Liimatainen, ACS Applied Materials & Interfaces 2017, 9, 25029.
T. Li, C. Chen, A. H. Brozena, J. Y. Zhu, L. Xu, C. Driemeier, J. Dai, O. J. Rojas, A. Isogai, L. Wågberg, L. Hu, Nature 2021, 590, 47.
Y. Y. Li, L. T. Cao, X. Yin, Y. Si, J. Y. Yu, B. Ding, Adv. Funct. Mater. 2020, 30, 1910426.
H. Liu, X. Y. Chen, Y. J. Zheng, D. B. Zhang, Y. Zhao, C. F. Wang, C. F. Pan, C. T. Liu, C. Y. Shen, Adv. Funct. Mater. 2021, 31, 2008006.
M. A. B. Meador, E. F. Fabrizio, F. Ilhan, A. Dass, G. H. Zhang, P. Vassilaras, J. C. Johnston, N. Leventis, Chem. Mater. 2005, 17, 1085.
M. Najberg, M. H. Mansor, T. Taille, C. Boure, R. Molina-Pena, F. Boury, J. L. Cenis, E. Garcion, C. Alvarez-Lorenzo, Carbohydr. Polym. 2020, 237, 116107.
C. C. Qian, L. H. Li, M. Gao, H. Y. Yang, Z. R. Cai, B. D. Chen, Z. Y. Xiang, Z. J. Zhang, Y. L. Song, Nano Energy 2019, 63, 103885.
L. Qiu, J. Z. Liu, S. L. Y. Chang, Y. Z. Wu, D. Li, Nat. Commun. 2012, 3, 1241.
W. Sakuma, S. Yamasaki, S. Fujisawa, T. Kodama, J. Shiomi, K. Kanamori, T. Saito, ACS Nano 2021, 15, 1436.
Y. Si, J. Y. Yu, X. M. Tang, J. L. Ge, B. Ding, Nat. Commun. 2014, 5, 5802.
Y. Si, X. Q. Wang, L. Y. Dou, J. Y. Yu, B. Ding, Science Advances 2018, 4, eaas8925.
J. W. Song, C. J. Chen, Z. Yang, Y. D. Kuang, T. Li, Y. J. Li, H. Huang, I. Kierzewski, B. Y. Liu, S. M. He, T. T. Gao, S. U. Yuruker, A. Gong, B. Yang, L. B. Hu, ACS Nano 2018, 12, 140.
M. Y. Song, J. G. Jiang, H. F. Qin, X. Y. Ren, F. Jiang, ACS Appl. Mater. Interfaces 2020, 12, 45363.
A. M. Tang, Y. J. Cui, T. T. Le, Applied Clay Science 2008, 41, 181.
D. Wang, H. Y. Peng, B. Yu, K. Q. Zhou, H. F. Pan, L. P. Zhang, M. Li, M. M. Liu, A. L. Tian, S. H. Fu, Chem. Eng. J. 2020, 389, 124449.
F. Wang, L. Y. Dou, J. W. Dai, Y. Y. Li, L. Q. Huang, Y. Si, J. Y. Yu, B. Ding, Angew. Chem. Int. Ed 2020, 59, 8285.
H. L. Wang, X. Zhang, N. Wang, Y. Li, X. Feng, Y. Huang, C. S. Zhao, Z. L. Liu, M. H. Fang, G. Ou, H. J. Gao, X. Y. Li, H. Wu, Sci. Adv. 2017, 3, e1603170.
J. M. Wang, D. Liu, Q. X. Li, C. Chen, Z. Q. Chen, P. G. Song, J. Hao, Y. W. Li, S. Fakhrhoseini, M. Naebe, X. G. Wang, W. W. Lei, ACS Nano 2019, 13, 7860.
L. Wang, M. Y. Zhang, B. Yang, J. J. Tan, X. Y. Ding, ACS Nano 2020, 14, 10633.
L. Y. Wang, X. Y. Zhang, Y. T. He, Y. Wang, W. Zhong, K. Mequanint, X. Z. Qiu, M. Xing, Adv. Funct. Mater. 2019, 29, 1806200.
S. Wang, X. Peng, L. Zhong, J. Tan, S. Jing, X. Cao, W. Chen, C. Liu, R. Sun, Journal of Materials Chemistry A 2015, 3, 8772.
Y. T. Wang, L. Y. Chen, H. Cheng, B. J. Wang, X. L. Feng, Z. P. Mao, X. F. Sui, Chemical Engineering Journal 2020, 402.
H. Y. Wu, Y. Y. Li, L. Zhao, S. Wang, Y. C. Tian, Y. Si, J. Y. Yu, B. Ding, ACS Appl. Mater. Interfaces 2020, 12, 27562.
Y. P. Wu, N. B. Yi, L. Huang, T. F. Zhang, S. L. Fang, H. C. Chang, N. Li, J. Oh, J. A. Lee, M. Kozlov, A. C. Chipara, H. Terrones, P. Xiao, G. K. Long, Y. Huang, F. Zhang, L. Zhang, X. Lepro, C. Haines, M. D. Lima, N. P. Lopez, L. P. Rajukumar, A. L. Elias, S. M. Feng, S. J. Kim, N. T. Narayanan, P. M. Ajayan, M. Terrones, A. Aliev, P. F. Chu, Z. Zhang, R. H. Baughman, Y. S. Chen, Nat. Commun. 2015, 6, 6141.
Z. Y. Wu, C. Li, H. W. Liang, J. F. Chen, S. H. Yu, Angewandte Chemie-International Edition 2013, 52, 2925.
Z. H. Zeng, E. Mavrona, D. Sacre, N. Kummer, J. M. Cao, L. A. E. Muller, E. Hack, P. Zolliker, G. Nystrom, ACS Nano 2021, 15, 7451.
Y. Zhao, C. G. Hu, Y. Hu, H. H. Cheng, G. Q. Shi, L. T. Qu, Angewandte Chemie-International Edition 2012, 51, 11371.
X. X. Zhang, M. H. Liu, H. K. Wang, N. Yan, Z. Y. Cai, Y. Yu, Carbohydrate Polymers 2019, 208, 232.
X. X. Zhang, F. Wang, L. Y. Dou, X. T. Cheng, Y. Si, J. Y. Yu, B. Ding, ACS Nano 2020, 14, 15616.
H. M. Zhang, J. L. Wang, G. B. Xu, Y. F. Xu, F. M. Wang, H. Shen, Journal of Hazardous Materials 2021, 406.
J. Y. Zhang, Y. H. Cheng, C. J. Xu, M. Y. Gao, M. F. Zhu, L. Jiang, Advanced Functional Materials 2021, 31, 2009349.

1. A method of preparing an aerogel, the method comprising:
freezing a suspension comprising nanofibrils and a solvent at a first temperature;
freeze-drying the frozen suspension to obtain sub-micron fibers;
freezing an aqueous suspension comprising the sub-micron fibers at a second temperature; and
freeze-drying the frozen suspension to obtain the aerogel.

2. The method of claim 1, wherein the solvent in the suspension comprising the nanofibrils is water, tert-butanol, dimethyl sulfoxide or mixtures thereof.

3. The method of claim 1 or 2, wherein the suspension comprising nanofibrils is an aqueous suspension comprising the nanofibrils in an amount of from about 0.01 wt % to about 1 wt %, based on the total weight of the aqueous suspension.

4. The method of claim 3, wherein the aqueous suspension comprises the nanofibrils in an amount of about 0.05 wt %, based on the total weight of the aqueous suspension.

5. The method of any one of claims 1 to 4, wherein the aqueous suspension comprising the sub-micron fibers comprises the sub-micron fibers in an amount of from about 0.01 wt % to about 2 wt %, based on the total weight of the aqueous suspension.

6. The method of claim 5, wherein the aqueous suspension comprises the sub-micron fibers in an amount of from about 0.2 wt % to about 2 wt %, based on the total weight of the aqueous suspension.

7. The method of claim 6, wherein the aqueous suspension comprises the sub-micron fibers in an amount of about 0.2 wt %.

8. The method of any one of claims 1 to 7, wherein the nanofibrils comprise cellulose, chitin, protein or combinations thereof.

9. The method of claim 8, wherein the nanofibrils comprise cellulose nanofibrils.

10. The method of claim 8 or 9, wherein the cellulose nanofibrils are prepared by a method comprising mechanical dispersion; enzymatic hydrolysis and mechanical dispersion; or chemical modification and mechanical dispersion.

11. The method of any one of claims 8 to 10, wherein the cellulose nanofibrils are oxidized cellulose nanofibrils prepared by a method comprising oxidizing a cellulose source and mechanically dispersing the oxidized cellulose source.

12. The method of claim 11, wherein the oxidation of the cellulose source comprises 2,2,6,6-tetramethylpiperidine 1-oxyl (TEMPO) oxidation.

13. The method of any one of claims 1 to 12, wherein the second temperature is greater than the first temperature.

14. The method of any one of claims 1 to 13, wherein the first temperature is in the range of from about −80° C. to about −200° C.

15. The method of claim 14, wherein the first temperature is about −196° C.

16. The method of any one of claims 1 to 15, wherein the second temperature is in a range of about −4° C. to about −50° C.

17. The method of claim 16, wherein the second temperature is about −20° C.

18. The method of any one of claims 1 to 17, wherein the method further comprises modifying a surface of the aerogel with a hydrophobic surface modifying agent.

19. The method of claim 18, wherein the modifying the surface comprises chemical vapor deposition of methyltrimethoxysilane.

20. The method of claim 18 or 19, wherein the modified surface is superhydrophobic.

21. An aerogel prepared by a method as defined in any one of claims 1 to 20.

22. An aerogel comprising a honeycomb structure, the cell walls of the honeycomb comprising interconnected sub-micron fibers.

23. The aerogel of claim 22, wherein the sub-micron fibers comprise cellulose, chitin, protein or combinations thereof.

24. The aerogel of claim 23, wherein the sub-micron fibers are cellulose sub-micron fibers.

25. The aerogel of claim 24, wherein the sub-micron fibers comprise oxidized cellulose.

26. The aerogel of any one of claims 22 to 25, wherein at least a portion of the cellulose has a cellulose Iβ crystal structure.

27. The aerogel of any one of claims 22 to 26, wherein a surface of the aerogel is modified with a hydrophobic surface modifying agent.

28. The aerogel of claim 27, wherein the hydrophobic surface modifying agent comprises methyltrimethoxysilane.

29. The aerogel of claim 27 or 28, wherein the modified surface is superhydrophobic.

30. The aerogel of any one of claims 22 to 29, wherein density of the aerogel is from about 2 mg/m³ to about 22 mg/m³.

31. The aerogel of any one of claims 22 to 30, wherein porosity of the aerogel is from about 98.7% to about 99.8%.

32. The aerogel of any one of claims 22 to 31, wherein the aerogel has isotropic superelasticity.

33. A use of an aerogel as defined in any one of claims 21 to 32 for oil-water separation, as an absorbent, as a thermal insulator, as an acoustic insulator, as a drug carrier, as a tissue scaffold, as an infrared shield and/or in a sensor.

34. A method of preparing sub-micron fibers comprising a cellulose Iβ crystal structure, the method comprising:
freezing a suspension comprising cellulose nanofibrils and a solvent at a temperature of from about-80°° C. to about-200° C.; and
freeze-drying the frozen suspension to obtain the sub-micron fibers.

35. A sub-micron fiber comprising a cellulose Iβ crystal structure prepared by a method as defined in claim 34.
